(19) United States
(12) Patent Application Publication
(10) Pub. No.: US 2023/0288403 A1
(43) Pub. Date: Sep. 14, 2023

(54) METHOD FOR INDUCING EXTRACELLULAR TRAP (ET) FORMATION

(71) Applicants: Tridek-One Therapeutics, Paris (FR); INSERM (Institut National de la Santé et de la Recherche Médicale), Paris (FR); Université Paris XIII, Villetaneuse (FR); Université Paris Cité, Paris (FR)

(72) Inventors: Giuseppina CALIGIURI, Paris (FR); Antonino NICOLETTI, Paris (FR); Thi Thu Trang TRAN, Paris (FR)

(73) Assignees: Tridek-One Therapeutics, Paris (FR); INSERM (Institut National de la Santé et de la Recherche Médicale), Paris (FR); Université Paris XIII, Villetaneuse (FR); Université Paris Cité, Paris (FR)

(21) Appl. No.: 18/167,285

(22) Filed: Feb. 10, 2023

(30) Foreign Application Priority Data
Feb. 10, 2022 (EP) 22 305 149.1

(51) Int. Cl. G01N 33/50

(52) U.S. Cl. CPC G01N 33/5032; G01N 33/5047

(57) ABSTRACT

An in vitro assay for inducing extracellular trap (ET) formation, including contacting immune cells, in in vitro culture or within a biological sample, with immobilized human polyvalent immunoglobulins; and to uses thereof, in particular in an in vitro method for screening a drug for its ability to modulate ET formation, in an in vitro method for assessing the susceptibility of a subject to ET formation, in an in vitro method for predicting the response of a subject to a modulator of ET formation, and in in vitro method for monitoring the response of a subject to an immunomodulator.

### FIELD

The present invention relates to the field of immunology and, more particularly, to the field of extracellular traps (ETs) released by leukocytes.

### BACKGROUND

Neutrophils represent up to 40 to 70% of leukocytes in humans and they are the first cell type recruited to the inflammatory site. As part of the innate immune system, they act as the first line of defense against pathogens. The mechanisms used by neutrophils to eliminate invading microorganisms include phagocytosis, reactive oxygen species (ROS) release and degranulation of microbicidal molecules. In 2004, an additional antimicrobial activity was identified: neutrophils are able to extrude a meshwork constituted of chromatin fibers, DNA, histones and granule-derived antimicrobial peptides and enzymes. These structures, called neutrophil extracellular traps (NETs), represent an important strategy to immobilize and kill pathogens.

Two mechanisms of NETs release have been identified: i) extrusion of mitochondrial DNA and serine protease from intact neutrophils, as opposed to ii) release of decondensed chromatin and granular contents into the extracellular space after dissolution of the nuclear and granular membranes, decondensation of the nuclear contents into the cytoplasm and rupture of the plasma membrane. The second mechanism is called NETosis and represents a distinct form of active cell death. NETosis can be triggered by diverse signals such as microbe-associated, inflammatory or endogenous molecules.

Even though NETs are involved in pathogen clearance, excessive NET formation can promote inflammation and tissue damage. A deleterious role of NETs has thus been identified, for example in chronic inflammatory diseases, as well as in cancer. Consequently, elucidation of the mechanisms behind NET formation, and more generally behind ET formation, has become an increasingly important topic.

Assays have thus been developed to study ET release, and in particular NET release, in vitro. The most frequently used compound in in vitro studies is phorbol 12-myristate 13-acetate (PMA), a synthetic activator of the ubiquitous signal transduction enzyme protein kinase C (PKC) which leads to the phosphorylation of gp91phox/Nox2 (NADPH oxidase 2). However, while PMA efficiently promotes ROS production and NET formation by neutrophils in vitro, ROS production and NET formation induced by PMA are not physiologically relevant, since they bypass membrane receptors and their downstream signaling intermediates. Both ROS production and NET formation induced by physiological stimuli depend on discrete signaling pathways involving Src family kinases and the spleen tyrosine kinase also known as SYK (van der Linden M et al. Sci Rep. 2017 Jul. 26; 7(1):6529), which can be activated by cell surface receptors, and in particular by the Fc receptor. Therefore, there is still a need to identify physiologically relevant inducers of ETs, in particular of NETs, to be used in in vitro assays.

The Inventors surprisingly discovered that immobilized human polyvalent immunoglobulins (also known as intravenous immunoglobulins or IVIg) are potent physiological inducers of ET formation by immune cells, and in particular of NET formation by neutrophils. Indeed, the data included hereinafter demonstrate that ET formation induced by immobilized human polyvalent immunoglobulins depends on SYK signaling. The present invention thus relates to an in vitro method for inducing ET formation, and in particular NET formation, by contacting immune cells (such as, for example, neutrophils in in vitro culture or immune cells comprised within a blood sample) with immobilized human polyvalent immunoglobulins, and to uses of said method.

### SUMMARY

The present invention relates to an in vitro method for inducing extracellular trap (ET) formation, said method comprising contacting immune cells, in in vitro culture or within a biological sample, with human polyvalent immunoglobulins, wherein said human polyvalent immunoglobulins are immobilized on a solid support. In some embodiments, the immune cells are granulocytes, macrophages and/or mast cells. In some embodiments, the immune cells are neutrophils and/or eosinophils. In some embodiments, the method does not comprise contacting the immune cells in in vitro culture or within a biological sample with phorbol-12-myristate-13-acetate (PMA).

In some embodiments, the solid support is coated with human polyvalent immunoglobulins using a coating solution comprising human polyvalent immunoglobulins at a concentration of at least about 3 μg/mL. In some embodiments, the immune cells are cultured at a concentration of at least about 1×10⁵ cells/mL of culture medium or present in the biological sample at a concentration of at least about 1×10⁵ cells/mL of sample.

In some embodiments, the immune cells are cultured in a culture medium comprising a cell-impermeant nucleic acid dye or the biological sample is diluted in a culture medium comprising a cell-impermeant nucleic acid dye. In some embodiments, the method comprises assessing ET formation by image analysis. In some embodiments, the cell-impermeant nucleic acid dye is fluorescent and the method comprises assessing ET formation by fluorescence detection.

The present invention also relates to an in vitro method for screening a drug for its ability to modulate extracellular trap (ET) formation, said method comprising:
a) contacting immune cells, in in vitro culture or within a biological sample, with a drug;
b) inducing ET formation by contacting the immune cells in in vitro culture or within a biological sample with human polyvalent immunoglobulins immobilized on a solid support according to the method described above; and
c) assessing the effect of the drug on ET formation in the in vitro culture of immune cells or in the biological sample,
   wherein the order of step a) and b) can be inverted.

The present invention also relates to an in vitro method for assessing extracellular trap (ET) formation in a subject, said method comprising:
a) contacting a biological sample comprising immune cells or an in vitro culture of immune cells, previously obtained from a subject, with human polyvalent immunoglobulins immobilized on a solid support, thereby inducing ET formation according to the method as described above; and
b) quantifying ET formation induced in the biological sample or in the in vitro culture of immune cells.

The present invention also relates to an in vitro method for assessing the susceptibility of a subject to extracellular trap (ET) formation, said method comprising:
a) contacting a biological sample comprising immune cells or an in vitro culture of immune cells, previously obtained from a subject, with human polyvalent immunoglobulins immobilized on a solid support, thereby inducing ET formation according to the method as described above; and
b) quantifying ET formation induced in the biological sample or in the in vitro culture of immune cells, thereby assessing the susceptibility of the subject to ET formation.

In some embodiments, the method for assessing the susceptibility of a subject to ET formation further comprises determining the ratio of immune cells:human polyvalent immunoglobulins required to induce maximal ET formation in the biological sample comprising immune cells or in the in vitro culture of immune cells.

The present invention also relates to an in vitro method for predicting the response of a subject to a modulator of extracellular trap (ET) formation, said method comprising:
a) contacting a biological sample comprising immune cells or an in vitro culture of immune cells, previously obtained from a subject, with a modulator of ET formation;
b) inducing ET formation by contacting the biological sample or the in vitro culture of immune cells with human polyvalent immunoglobulins immobilized on a solid support according to the method as described above; and
c) assessing the effect of the modulator of ET formation on ET formation in the biological sample or in the in vitro culture of immune cells, wherein the order of step a) and b) can be inverted.

In some embodiments, the method for predicting the response of a subject to a modulator of ET formation further comprises a step of comparing the ET formation induced in the biological sample or in the in vitro culture of immune cells to a reference value, wherein the reference value is the ET formation induced in a biological sample comprising immune cells or in an in vitro culture of immune cells, previously obtained from the subject, without the modulator of ET formation.

The present invention further relates to an in vitro method for monitoring the response of a subject to a therapeutic or prophylactic agent, said method comprising:
a) contacting a biological sample comprising immune cells or an in vitro culture of immune cells, previously obtained from a subject, with human polyvalent immunoglobulins immobilized on a solid support, thereby inducing ET formation according to the method as described above, wherein said subject was treated or is being treated with a therapeutic or prophylactic agent;
b) quantifying ET formation induced in the biological sample or in the in vitro culture of immune cells; and
c) comparing the ET formation induced in the biological sample or in the in vitro culture of immune cells to a reference value, thereby monitoring the response of the subject to the therapeutic or prophylactic agent.
   In some embodiments, the reference value is the ET formation induced in a biological sample comprising immune cells or in an in vitro culture of immune cells, previously obtained from the subject before treatment with the therapeutic or prophylactic agent.

In some embodiments, the subject is suffering from a disease or condition to which ET formation may contribute, in particular selected from an inflammatory disease, a thromboembolic disease, a cancer (such as a solid cancer, a blood cancer, or a lymphoma), and a fibrosis. In some embodiments, the disease or condition to which ET formation may contribute is selected from vasculitis, in particular ANCA vasculitis; asthma; rheumatoid arthritis (RA); chronic obstructive pulmonary disease (COPD); systemic lupus erythematous (SLE); systemic sclerosis; adverse cardiovascular events such as myocardial infarction; ischemic stroke; atherosclerosis; venous thrombosis; cancer, in particular solid cancer; fibrosis, for example cystic fibrosis; sepsis; gout; and Alzheimer's disease.

### BRIEF DESCRIPTION OF THE DRAWINGS

FIGS. 1A to 1C are images obtained with fluorescence microscopy showing NET formation by freshly isolated human peripheral blood neutrophils cultured in vitro in presence of the cell-impermeable nucleic acid dye Sytox Green. 1A shows human neutrophils cultured in plates coated with human polyvalent immunoglobulins (obtained with a coating solution comprising human polyvalent Ig at a concentration of 100 μg/mL). 1B shows human neutrophils cultured in plates coated with human serum albumin or HSA (obtained with a coating solution comprising HSA at a concentration of 100 μg/mL), used as negative control. 1C shows human neutrophils cultured with PMA (25 nM), used as positive control.

FIGS. 2A to 2H are images obtained with live cell fluorescent microscopy showing NET formation over a 24-hour culture period by freshly isolated human peripheral blood neutrophils cultured in vitro in plates coated with human polyvalent Ig (obtained with a coating solution comprising human polyvalent Ig at a concentration of 100 μg/mL). NET formation is shown after 3-, 6-, 9-, 12-, 15-, 18-, 21- and 24-hour culture in presence of the cell-impermeable nucleic acid dye Sytox Green. 2A shows 3-hour culture. 2B shows 6-hour culture. 2C shows 9-hour culture. 2D shows 12-hour culture. 2E shows 15-hour culture. 2F shows 18-hour culture. 2G shows 21-hour culture. 2H shows 24-hour (1 day) culture.

FIGS. 3A to 3H are images obtained with live cell fluorescent microscopy showing NET formation over a 24-hour culture period by freshly isolated human peripheral blood neutrophils cultured in vitro with PMA (25 nM). NET formation is shown after 3-, 6-, 9-, 12-, 15-, 18-, 21- and 24-hour culture in presence of the cell-impermeable nucleic acid dye Sytox Green. 3A shows 3-hour culture. 3B shows 6-hour culture. 3C shows 9-hour culture. 3D shows 12-hour culture. 3E shows 15-hour culture. 3F shows 18-hour culture. 3G shows 21-hour culture. 3H shows 24-hour (1 day) culture.

FIGS. 4A and 4B are graphs showing the NET formation time-course (4A) and the NET formation rate change (4B) over a 24-hour culture period of freshly isolated human peripheral blood neutrophils cultured in vitro in presence of the cell-impermeable nucleic acid dye Sytox Green, either with plate-bound human polyvalent Ig (obtained with a coating solution comprising human polyvalent Ig at a concentration of 100 μg/mL), plate-bound human serum albumin (negative control, obtained with a coating solution comprising HSA at a concentration of 100 μg/mL), or 25 nM PMA (positive control). NET formation is assessed at 0-, 3-, 6-, 9-, 12-, 15-, 18-, 21-, and 24-hour culture. Data are expressed as integrated fluorescence intensity (green calibrated unit or GCU)μ²/image/well.

FIGS. 5A and 5B are graphs comparing the formation of NETs by freshly isolated human peripheral blood neutrophils cultured in vitro in presence of the cell-impermeable nucleic acid dye Sytox Green, either with soluble human polyvalent Ig (soluble IVIG—5A) or with plate-bound human polyvalent Ig (immobilized IVIG—5B). Human polyvalent Ig are used at the indicated concentration (concentration in the coating solution for the plate-bound human polyvalent Ig). Data correspond to integrated green fluorescent intensity expressed as GCU/well.

FIGS. 6A to 6E are a combination of graphs showing the production of ROS (arbitrary units), detected through the use of an oxidative-sensitive fluorescent probe, and the formation of NETs (arbitrary units), detected through the use of the cell-impermeable nucleic acid dye Sytox Green, over a 5-h time period by freshly isolated human peripheral blood neutrophils cultured in vitro in plates coated with human polyvalent Ig (or IVIg). Data are expressed as GCU/well. Increasing densities of plate-bound human polyvalent Ig are used, obtained with a coating solution comprising human polyvalent Ig at a concentration ranging from 3 to 300 μg/mL. 6A corresponds to deposits of IVIg obtained with a coating solution comprising IVIg at a concentration of 3 μg/mL. 6B corresponds to deposits of IVIg obtained with a coating solution comprising IVIg at a concentration of 10 μg/mL. 6C corresponds to deposits of IVIg obtained with a coating solution comprising IVIg at a concentration of 30 μg/mL. 6D corresponds to deposits of IVIg obtained with a coating solution comprising IVIg at a concentration of 100 μg/mL. 6E corresponds to deposits of IVIg obtained with a coating solution comprising IVIg at a concentration of 300 μg/mL.

FIGS. 7A and 7B are graphs showing the production of ROS (CM-H₂DCFDA—7A) and the formation of NETs (Sytox Green—7B) over a period of 6 hours (0-360 min) by freshly isolated human peripheral blood neutrophils cultured in vitro in plates coated with human polyvalent Ig (obtained with a coating solution comprising human polyvalent Ig at a concentration of 100 μg/mL) in the presence of different concentrations of the SYK inhibitor (or SYKi) BAY-61-3606 (0, 0.05, 0.1, 0.5, 1, 5 as indicated). Data are expressed as GCU/well.

FIG. 8 is a graph showing the production of ROS (CM-H₂DCFDA) over a period of 3 hours (0-180 minutes) by freshly isolated human peripheral blood neutrophils cultured in vitro in plates coated with human polyvalent Ig (obtained with a coating solution comprising human polyvalent Ig at a concentration of 100 μg/mL) in the presence of different concentrations of the calcineurin inhibitor FK506, also known as tacrolimus (0.3, 1, 3, 10, 30, 100 nM, as indicated). Data are expressed as GCU/well.

FIGS. 9A and 9B are graphs comparing the formation of NETs by freshly isolated human peripheral blood neutrophils cultured in vitro in presence of the cell-impermeable nucleic acid dye Sytox Green with soluble motavizumab IgG4 (used at increased concentration ranging from 1 to 100 μg/mL, as indicated) either with plate-bound human polyvalent Ig (immobilized IVIG—9A) or without plate-bound human polyvalent Ig (without immobilized IVIG—9B). The SYK inhibitor BAY-61-3606 (5 μM) is used as a control (SYKi) in combination with immobilized human polyvalent Ig (9B) or in combination with both immobilized human polyvalent Ig and 100 μg/mL of soluble motavizumab IgG4 (9A). When present, deposits of human polyvalent Ig are obtained with a coating solution comprising human polyvalent Ig at a concentration of 100 μg/mL. Data are expressed as GCU/well.

FIGS. 10A to 10C, 11A to 11C, 12A to 12C, 13A to 13C, 14A to 14C, 15A to 15C, and 16A to 16C are combinations of graphs showing the formation of NETs (arbitrary units—AUC) by human neutrophils freshly isolated from samples of peripheral blood of 7 healthy individuals (referred to as 85, 86, 87, 88, 89, 90, and 91) as a function of the density of human polyvalent Ig deposits. For each individual, neutrophils were isolated from samples of peripheral blood obtained at three independent time points (time points 1, 2, and 3), separated by a least two weeks. For each individual, isolated neutrophils were cultured in presence of the cell-impermeable nucleic acid dye Sytox Green with increasing densities of human polyvalent Ig obtained with a coating solution comprising human polyvalent Ig at a concentration of 3, 10, 30, 100 or 300 μg/mL. 10A-10C show donor 86 at time point 1 (10A), time point 2 (10B) and time point 3 (10C). 11A-11C shows donor 87 at time point 1 (11A), time point 2 (11B) and time point 3 (11C). 12A-12C shows donor 88 at time point 1 (12A), time point 2 (12B) and time point 3 (12C). 13A-13C shows donor 89 at time point 1 (13A), time point 2 (13B) and time point 3 (13C). 14A-14C shows donor 90 at time point 1 (14A), time point 2 (14B) and time point 3 (14C). 15A-15C shows donor 85 at time point 1 (15A), time point 2 (15B) and time point 3 (15C). 16A-16C shows donor 91 at time point 1 (16A), time point 2 (16B) and time point 3 (16C).

FIG. 17 is a graph showing the formation of extracellular traps (ETs) by freshly isolated human peripheral blood eosinophils cultured in vitro in presence of the cell-impermeant nucleic acid dye Sytox Green either with increasing concentration of plate-bound human polyvalent Ig or with coated bovine serum albumin (Control). The SYK inhibitor BAY-61-3606 (5 μM) is used as a control (SYKi) in combination with immobilized human polyvalent Ig as indicated. Human polyvalent Ig are used at the indicated concentration (concentration in the coating solution for the plate-bound human polyvalent Ig). Data correspond to integrated green fluorescent intensity expressed as GCU/well.

FIG. 18 is a graph showing the formation of extracellular traps (ETs) by fresh human peripheral whole blood incubated in vitro in presence of the cell-impermeant nucleic acid dye Sytox Green either with increasing concentration of plate-bound human polyvalent Ig or with coated bovine serum albumin (Control). The SYK inhibitor BAY-61-3606 (5 μM) is used as a control (SYKi) in combination with immobilized human polyvalent Ig as indicated. Human polyvalent Ig are used at the indicated concentration (concentration in the coating solution for the plate-bound human polyvalent Ig). Data correspond to integrated green fluorescent intensity expressed as GCU/well.

### DETAILED DESCRIPTION

In the present invention, the following terms have the following meanings:

“Ig” refers to immunoglobulin(s).

“About” preceding a figure encompasses plus or minus 10%, or less, of the value of said figure. It is to be understood that the value to which the term “about” refers is itself also specifically, and preferably, disclosed.

“Biological sample” refers to a cell, tissue or organ. Examples of biological samples include bodily fluids (preferably blood), whole blood, apheresis blood, serum, plasma, urine, feces, synovial fluid, bronchoalveolar lavage fluid, sputum, lymph, ascitic fluids, urine, amniotic fluid, peritoneal fluid, cerebrospinal fluid, pleural fluid, pericardial fluid, and alveolar macrophages, tissue lysates, biopsies and extracts prepared from diseased tissues. Preferably, as used herein, a biological sample comprises immune cells, such as, for example, neutrophils.

“DNA” refers to deoxyribonucleic acid.

“Fc” or “fragment crystallizable” refers to the tail region of an immunoglobulin that interacts with cell surface receptors called Fc receptors.

“Measuring” or “measurement”, or alternatively “detecting” or “detection”, mean assessing the presence, absence, level, or quantity of a given object, e.g., ET or NET formation. “Measuring” or “measurement”, or alternatively “detecting” or “detection” as used herein include the derivation of the qualitative or quantitative assessment of ET or NET formation.

“Subject” refers to an animal, preferably a mammal, more preferably a human. In some embodiments, a subject may be a “patient”, who/which is awaiting the receipt of, or is receiving medical care or was/is/will be the object of a medical procedure, or is monitored for the development of the targeted disease or condition, such as, for example, a neutrophil-associated disease or condition, in particular a disease or condition to which NET formation may contribute. In some embodiments, the subject is an adult (for example a subject above the age of 18, 19, 20, or 21). In some embodiments, the subject is a child (for example a subject below the age of 21, 20, 19, or 18). In some embodiments, the subject is a male. In some embodiments, the subject is a female. In some embodiments, the subject is affected, preferably is diagnosed, with a neutrophil-associated disease or condition, in particular with a disease or condition to which NET formation may contribute. In some embodiments, the subject is at risk of developing a neutrophil-associated disease or condition, in particular with a disease or condition to which NET formation may contribute. Examples of risks factor include genetic predisposition, susceptibility to NET formation, or familial history of neutrophil-associated diseases.

“Treating” or “treatment” or “alleviation” refers to a therapeutic treatment, to a prophylactic (or preventative) treatment, or to both a therapeutic treatment and a prophylactic (or preventative) treatment; wherein the object is to prevent or slow down (lessen) the targeted pathologic condition or disorder. Those in need of treatment include those already with the disorder as well as those prone to have the disorder or those in whom the disorder is to be prevented. In some embodiments, a subject is successfully “treated” for a disease or disorder if, after receiving a therapeutic amount of a therapeutic agent, such as a NETosis modulator or an immunomodulator, the subject shows at least one of the following: relief to some extent of one or more of the symptoms associated with the disease or disorder to be treated, and/or improvement in quality-of-life issues. The above parameters for assessing successful treatment and improvement in the disease or disorder are readily measurable by routine procedures familiar to a physician.

The present invention firstly relates to an in vitro method for inducing extracellular trap (ET) formation, comprising contacting cells in in vitro culture with human polyvalent immunoglobulins immobilized on a solid support. The present invention also relates to an in vitro method for inducing extracellular trap (ET) formation, comprising contacting a biological sample, such as a blood sample, with human polyvalent immunoglobulins immobilized on a solid support.

“Extracellular Traps” or “ETs” refer to lacy structures constituted of a DNA backbone embedded with antimicrobial peptides, proteases and histones. Extracellular traps can be released by immune cells (i.e., innate immune cells or adaptive immune cells), including neutrophils, eosinophils, basophils, macrophages, mast cells, plasmacytoid dendritic cells, B lymphocytes and T lymphocytes. Extracellular trap release allows to immobilize and kill invading microorganisms in the extracellular milieu. Extracellular trap formation can be of two types: with or without the death of the producer cell. The cell death program associated with extracellular traps formation is called ETosis.

Another object of the present invention is an in vitro method for inducing ETosis, comprising contacting cells in in vitro culture with human polyvalent immunoglobulins immobilized on a solid support. The present invention also relates to an in vitro method for inducing ETosis, comprising contacting a biological sample, such as a blood sample, with human polyvalent immunoglobulins immobilized on a solid support.

In some embodiments, the in vitro method of the present invention for inducing extracellular trap (ET) formation, comprising contacting cells in in vitro culture, or a biological sample comprising cells, with human polyvalent immunoglobulins immobilized on a solid support, also induces ETosis.

As indicated above, ET release and/or ETosis can be induced in a number of immune cells, in particular in immune cells expressing Fc receptors at their surface, such as granulocytes (e.g., neutrophils, eosinophils, basophils), macrophages, mast cells, dendritic cells (in particular plasmacytoid dendritic cells), B lymphocytes (or B cells) and T lymphocytes (or T cells).

“Granulocytes” refers to specific leukocytes characterized by the presence of cytoplasmic granules containing lytic enzymes and a poly-lobed nucleus. Granulocytes represent the most abundant cell type in human peripheral blood, belong to the myeloid cell family, and are part of the innate immune system. Depending on the content of their cytoplasmic granules, granulocytes are classified as neutrophils, eosinophils or basophils. Granulocytes are important in the removal of bacteria and parasites from the body.

“Neutrophils”, “Polymorphonuclear neutrophils (PMNs)” or “Neutrophilic granulocytes” refer to the most abundant type of granulocytes in human. Neutrophils are the first type of leukocytes recruited at the injury or infection sites, in response to CXCL8 produced by stressed epithelial cells and tissue-resident macrophages. Neutrophils represent the first line of defense against invading pathogens, by phagocytosis of microorganisms, release of antimicrobial factors contained in cytoplasmic granules, production of reactive oxygen species or extrusion of neutrophil extracellular traps (NETs). Neutrophils' granules contain bactericidal enzymes such as lysozyme, myeloperoxidase, neutral proteases, acid hydrolases, collagenase, lactoferrin and cathepsin B, which possess potent antimicrobial activity but are also highly cytotoxic.

“Eosinophils”, “Polymorphonuclear eosinophils” or “Eosinophilic granulocytes” refer to a variety of granulocytes implicated in host defense against nematodes and other parasitic infections thanks to their capacities of phagocytosis, degranulation and reactive oxygen species (ROS) production. Additionally, eosinophils have been shown to release eosinophil extracellular traps (EETs). However, eosinophils' degranulation capacity can also be detrimental and participate in the inflammatory process of allergic diseases.

“Basophils”, “Polymorphonuclear basophils” or “Basophilic granulocytes” refer to the least abundant type of granulocytes, which possesses a beneficial role in infections with helminth parasites. Basophils have important roles in the regulation of adaptive immune responses through their secretion of various cytokines, but also their capacity of acting as antigen-presenting cells. Basophils have also been shown to release basophil extracellular traps (BETs).

“Macrophages” are tissue-resident leukocytes involved in innate immune defense. They possess phagocytosis abilities, which allows them to eliminate pathogens and remove cell debris, and are involved in T lymphocytes activation initiation. An additional effector function has recently been described for macrophages: the ability to release macrophage extracellular traps (METs) composed of DNA, histones, myeloperoxidase and lysozymes.

“Mast cells” are tissue-resident leukocytes characterized by a monolobulated nucleus and granules containing histamine, heparin, tryptase and chymase. Mast cells are involved in immune responses to various pathogens and have been shown to have a role in allergy. Mast cells are also able to release mast cell extracellular traps (MCETs) comprising DNA, tryptase, histones and cathelicidins.

“Dendritic cells” or “DC” account for less than 1% of leukocytes in peripheral blood. Dendritic cells play a role in linking the innate and adaptive immune systems. They are sentinels in peripheral tissues, patrolling the presence of antigens for presentation to T lymphocytes. Dendritic cells, and in particular plasmacytoid dendritic cells (pDCs), have been recently shown to release dendritic cell extracellular traps (DCETs).

“B lymphocytes” or “B cells” represent about 15% of peripheral blood leukocytes and are involved in humoral immunity through their production of antibodies. B lymphocytes also have the ability to present antigens and produce cytokines. More recently, B lymphocytes have been shown to produce lymphocyte extracellular traps (LETs).

“T lymphocytes” or “T cells” are a specific type of leukocytes possessing important functions in directly killing infected host cells (i.e., cytotoxic T lymphocytes), activating other immune cells, producing cytokines and regulating the immune response (i.e., helper T lymphocytes). Both cytotoxic and helper T lymphocytes have been shown to produce lymphocyte extracellular traps (LETs).

The cells to be contacted with human polyvalent immunoglobulins immobilized on a solid support according to the method described herein may be any immune cell, in particular any cell expressing Fc receptors at its surface. For example, said cells may be neutrophils, eosinophils, basophils, macrophages, mast cells, dendritic cells, B lymphocytes and/or T lymphocytes. Thus, in some embodiments, said cells are immune cells. In some embodiments, said immune cells are granulocytes (i.e., neutrophils, eosinophils, and/or basophils), macrophages and/or mast cells. In some embodiments, said immune cells are granulocytes (i.e., neutrophils, eosinophils, and/or basophils). In some embodiments, said immune cells are neutrophils and/or eosinophils. In some embodiments, said immune cells are neutrophils.

In some embodiments, the cells, preferably neutrophils, are human cells.

In some embodiments, the cells, in particular neutrophils, are comprised within a biological sample obtained from a subject. In some embodiments, the cells, preferably neutrophils, are isolated from a biological sample obtained from a subject. In some embodiments, the cells, preferably neutrophils, are comprised within or isolated from a blood sample. As used herein, “blood” includes whole blood, plasma, serum, constituents, or any other derivative of blood. Thus, a blood sample may be a whole blood sample. In some embodiments, the cells, preferably neutrophils, are comprised within or isolated from a whole blood sample. In some embodiments, the cells, preferably neutrophils, are comprised within or isolated from a peripheral blood sample.

In some embodiments, the cells comprised within a biological sample obtained from a subject are not isolated from said biological sample. Thus, in some embodiments, a biological sample (such as, for example, a blood sample) obtained from a subject is diluted in culture medium as described herein. The dilution may be a 0.1% dilution (1/1000 dilution), a 1% dilution (1/100 dilution), a 5% dilution (1/20 dilution), a 10% dilution (1/10 dilution), a 20% dilution (1/5 dilution), a 25% dilution (1/4 dilution), or 50% dilution (1/2 dilution). In some embodiments, the biological sample obtained from a subject is diluted in culture medium as described herein at a dilution ranging from 1/200 dilution to 1/2 dilution.

In some embodiments, in the methods described herein, the biological sample obtained from a subject is diluted in culture medium as described above and contacted with human polyvalent immunoglobulins immobilized on a solid support as described herein.

In some embodiments, the cells, preferably neutrophils, are comprised within or isolated from a biological sample as described above previously taken from a subject, i.e., the methods of the present invention do not comprise an active step of recovering a sample from a subject. Consequently, according to some embodiments, the methods described herein are non-invasive methods, i.e., the methods described herein are in vitro methods.

Methods used for isolating cells from a biological sample, in particular a blood sample, are well-known in the art. Methods used for isolating neutrophils from a biological sample, in particular a blood sample, are well-known in the art. For example, a standard density gradient separation method to isolate human neutrophils from whole blood is described in Oh et al., J Vis Exp. 2008, 23; (17):745. Additionally, commercial kits are available, such as: EasySep™ Direct Human Neutrophil Isolation Kit (Catalog #19666, Stem Cell Technologies), MACSxpress® Whole Blood Neutrophil Isolation Kit, human (Catalog #130-104-434, Miltenyi Biotec).

The present invention further relates to an in vitro method for inducing neutrophil extracellular trap (NET) formation, comprising contacting neutrophils in in vitro culture with human polyvalent immunoglobulins immobilized on a solid support. The present invention also relates to an in vitro method for inducing NET formation, comprising contacting a biological sample comprising neutrophils with human polyvalent immunoglobulins immobilized on a solid support.

“Neutrophil Extracellular Traps” or “NETs” refer to complexes comprising large strands of decondensed DNA, histones and granular proteins which are released by neutrophils. The primary function of NETs is the trapping of extracellular microorganisms. NETs capture bacteria and fungi while simultaneously providing high local concentrations of anti-microbial components, thus killing pathogens extracellularly. NET formation can be triggered by various molecular mechanisms including receptor-mediated signals (such as TLR engagement), or calcium channel formation through ionophores. NETs are also highly toxic to the host and NET release contributes to cellular injury and organ dysfunction.

Another object of the present invention is an in vitro method for inducing NETosis, comprising contacting neutrophils in in vitro culture with human polyvalent immunoglobulins immobilized on a solid support. The present invention also relates to an in vitro method for inducing NETosis, comprising contacting a biological sample comprising neutrophils with human polyvalent immunoglobulins immobilized on a solid support.

In some embodiments, the in vitro method of the present invention for inducing NET formation, comprising contacting neutrophils in in vitro culture, or a biological sample comprising neutrophils, with human polyvalent immunoglobulins immobilized on a solid support, also induces NETosis.

“NETosis” refers to the specific cell death program which neutrophils may undergo following NET formation. After receiving a signal inducing NET formation, a cascade of events is initiated, leading to chromatin decondensation, nuclear membrane disintegration, mixing of decondensed DNA with granules and cytosolic proteins and perforation of the plasma membrane. Finally, meshes composed of nuclear material, associated with cytoplasmic and nuclear proteins are extruded by neutrophils in the extracellular space.

The cells, preferably neutrophils, to be contacted with human polyvalent immunoglobulins immobilized on a solid support according to the method described herein may be cultured in any suitable culture medium. Suitable culture media are well-known to one skilled in the art and may be selected, for example, depending on the cells. In particular, suitable culture media include any isotonic medium comprising calcium and magnesium. Examples of suitable media include HBSS (Hank's Buffered Saline Solution) medium, RPMI (Roswell Park Memorial Institute) 1640 medium, and DMEM (Dulbecco's Modified Eagle Medium). The culture medium may be supplemented with additional substances such as serum and serum components, vitamins, reducing agents, and/or buffering agents.

In some embodiments, the cells, preferably neutrophils, are cultured in HBSS medium containing calcium and magnesium (said medium is commercially available, for example from Gibco (reference 24020-091)).

The cells, preferably neutrophils, may be cultured under any suitable culture conditions, for example with regards to temperature, humidity, CO₂. Suitable culture conditions are well-known to one skilled in the art and may be selected, for example, depending on the cells. In some embodiments, the cells, preferably neutrophils, are cultured in culture conditions suitable for human cells. In some embodiments, the cells, preferably neutrophils, are cultured at 37° C. and 5% CO₂.

In some embodiments, the cells, preferably neutrophils, are cultured for at least 3 hours. In some embodiments, the cells, preferably neutrophils, are cultured for at least 4, 5, 6, 7, 8, 9, 10, 12, 15, 18, or 24 hours.

Advantageously, the methods described herein do not involve contacting cells, preferably neutrophils, with phorbol-12-myristate-13-acetate (PMA). Therefore, in some embodiments, the methods described herein do not comprise contacting the cells, preferably neutrophils, in in vitro culture with PMA. Accordingly, in some embodiments, the methods described herein do not comprise contacting a biological sample, such as a blood sample, with PMA.

In some embodiments, the cells, preferably neutrophils, are cultured at a concentration of at least about 1×10⁴, 5×10⁴, 1×10⁵, 5×10⁵, 1×10⁶, 5×10⁶, 1×10⁷, or 5×10⁷ cells per milliliter of culture medium, preferably at a concentration of at least about 1×10⁶ cells/mL of culture medium. In some embodiments, the cells, preferably neutrophils, are cultured at a concentration ranging from about 1×10⁴ to about 5×10⁷ cells/mL of culture medium, preferably from about 1×10⁵ to about 1×10⁷ cells/mL of culture medium, more preferably from about 5×10⁵ to about 5×10⁶ cells/mL of culture medium. In some embodiments, the cells, preferably neutrophils, are cultured at a concentration of about 1×10⁴, 5×10⁴, 1×10⁵, 5×10⁵, 1×10⁶, 5×10⁶, 1×10⁷, or 5×10⁷ cells/mL of culture medium, preferably at a concentration of 1×10⁶ cells/mL of culture medium.

In some embodiments, the cells, preferably neutrophils, are cultured at a density of at least about 5×10³, 7.5×10³, 1×10⁴, 2.5×10⁴, 5×10⁴, 7.5×10⁴, 1×10⁵, 2.5×10⁵, 5×10⁵, 7.5×10⁵, or 1×10⁶ cells per well in a 96-well culture plate, preferably at a density of at least about 7.5×10⁴ cells per well in a 96-well culture plate. In some embodiments, the cells, preferably neutrophils, are cultured at a density ranging from about 5×10³ to about 5×10⁵ cells per well in a 96-well culture plate, preferably from about 7.5×10³ to about 2.5×10⁵ cells per well in a 96-well culture plate, more preferably from about 1×10⁴ to about 1×10⁵ per well in a 96-well culture plate. In some embodiments, the cells, preferably neutrophils, are cultured at a density of about 5×10³, 7.5×10³, 1×10⁴, 2.5×10⁴, 5×10⁴, 7.5×10⁴, 1×10⁵, 2.5×10⁵, 5×10⁵, 7.5×10⁵, or 1×10⁶ cells per well in a 96-well culture plate, preferably at a density of about 7.5×10⁴ cells per well in a 96-well culture plate.

In some embodiments, the cells, preferably neutrophils, are cultured at a concentration or density equivalent to a density of at least about 5×10³, 7.5×10³, 1×10⁴, 2.5×10⁴, 5×10⁴, 7.5×10⁴, 1×10⁵, 2.5×10⁵, 5×10⁵, 7.5×10⁵, or 1×10⁶ cells per well in a 96-well culture plate, preferably at a concentration or density equivalent to a density of at least about 7.5×10⁴ cells per well in a 96-well culture plate. In some embodiments, the cells, preferably neutrophils, are cultured at a concentration or density equivalent to a density ranging from about 5×10³ to about 5×10⁵ cells per well in a 96-well culture plate, preferably from about 7.5×10³ to about 2.5×10⁵ cells per well in a 96-well culture plate, more preferably from about 1×10⁴ to about 1×10⁵ per well in a 96-well culture plate. In some embodiments, the cells, preferably neutrophils, are cultured at a concentration or density equivalent to a density of about 5×10³, 7.5×10³, 1×10⁴, 2.5×10⁴, 5×10⁴, 7.5×10⁴, 1×10⁵, 2.5×10⁵, 5×10⁵, 7.5×10⁵, or 1×10⁶ cells per well in a 96-well culture plate, preferably at a concentration or density equivalent to a density of about 7.5×10⁴ cells per well in a 96-well culture plate. In some embodiments, a density of about 7.5×10⁴ cells per well in a 96-well culture plate is equivalent to a concentration of about 1×10⁶ cells per milliliter of culture medium.

“Polyvalent immunoglobulins” or “intravenous immunoglobulins” or “IVIg” refers to preparations of pooled normal polyspecific immunoglobulins, mostly IgG, obtained from large numbers of donors. IVIg thus correspond to a mix of immunoglobulins extracted and usually prepared from the plasma of between 1,000 and 15,000 donors. IVIg preparations consist of polyclonal natural immunoglobulins secreted by plasma B cells in response to immune stimuli. IVIg may comprise immunoglobulins secreted in response to microbial antigens, self-antigens, and/or anti-idiotypic antibodies which recognize other immunoglobulins. Clinically IVIg are widely used in replacement therapy in primary immunodeficiency syndromes and in secondary immunodeficiencies, as well as for the prevention and treatment of infectious diseases. Furthermore, IVIg are also used for immune modulation of patients with autoimmune and immune-complex diseases. IVIg are commercially available from several sources, for example from CSL Behring (King of Prussia, Pa., USA), Bio Products Laboratory (Elstree, United Kingdom), Octapharma (Lachen, Switzerland), LFB (les Ulis, France), Kedrion Biopharma Inc. (Barga, Italy), or Takeda Pharmaceuticals (Tokyo, Japan). In some embodiments, the IVIg are prepared from human donors. Therefore, in some embodiments, the polyvalent immunoglobulins are human polyvalent immunoglobulins.

In some embodiments, the IVIg are a 100 mg/mL solution for infusion, for example obtained from Privigen (CSL Behring UK Limited).

The solid support on which the IVIg are immobilized may be any vessel suitable for cell culture. Thus, in some embodiments, the solid support is a cell culture vessel (or culture vessel), in particular a sterile cell culture vessel. Examples of cell culture vessels include cell culture dishes, cell culture plates, cell culture flasks, cell culture bottles, cell culture tubes. In some embodiments, the solid support is a cell culture plate (or culture plate). In some embodiments, the solid support is a cell culture dish (or culture dish).

In some embodiments, the solid support is a multiple-well cell culture plate. Examples of multiple-well cell culture plates include 4-well, 6-well, 8-well, 12-well, 24-well, 48-well, and 96-well cell culture plates. In some embodiments, the solid support is a 4-well, 6-well, 8-well, 12-well, 24-well, 48-well, or a 96-well cell culture plate.

In some embodiments, the solid support is a culture vessel, for example a culture plate, suitable for fluorescence microscopy. In some embodiments, the solid support is a culture vessel, for example a culture plate, suitable for live fluorescence microscopy.

In some embodiments, the solid support has been exposed to a plasma gas in order to increase its hydrophobicity. In some embodiments, the solid support has increased protein binding capacity. In some embodiments, the solid support is high-binding. In some embodiments, high-binding corresponds to a binding capacity of about 400 to 500 ng IgG/cm2.

In some embodiments, the solid support is a high-binding, sterile 96-well plate suitable for fluorescence microscopy.

The solid support on which the IVIg are immobilized may also be any beads suitable for cell culture, in particular for culture of neutrophils. Thus, in some embodiments, the solid support is beads, preferably phagocytosis-resistant beads. By phagocytosis-resistant, it is meant that the beads cannot be taken in by the cells, in particular neutrophils, via phagocytosis. In some embodiments, the beads have a diameter greater than about 30 μm. In some embodiments, the beads are polystyrene beads. In some embodiments, the beads are epoxy beads.

In some embodiments, the solid support as described above is coated with human polyvalent immunoglobulins. Thus, in some embodiments, the solid support as described above is or was previously treated with a coating solution comprising human polyvalent immunoglobulins in order to obtain a solid support coated with human polyvalent immunoglobulins.

Accordingly, in some embodiments, the method as described above further comprises a first step of treating a solid support with a coating solution comprising human polyvalent immunoglobulins in order to obtain a solid support coated with human polyvalent immunoglobulins, that it to say a solid support on which human polyvalent immunoglobulins are immobilized.

Thus, in some embodiments, the in vitro method for inducing ET formation, preferably NET formation, and/or for inducing ETosis, preferably NETosis, comprises:
treating a solid support as described above with a coating solution comprising human polyvalent immunoglobulins; and
contacting cells, preferably neutrophils, in in vitro culture or comprised within a biological sample, with the solid support coated with human polyvalent immunoglobulins.

In some embodiments, the solid support is treated with a coating solution comprising human polyvalent immunoglobulins for about 1, 2, 3, 4, or 5 hours, preferably for about 2 hours. In some embodiments, the solid support is treated with a coating solution comprising human polyvalent immunoglobulins at room temperature.

The coating solution may be any suitable buffer solutions, in particular buffer solutions suitable for cell culture. Examples of buffer solutions include PBS (phosphate buffer saline). In some embodiments, the coating solution is PBS. In some embodiments, the coating solution is DPBS (Dulbecco's Phosphate—Buffered Saline) containing no calcium and no magnesium (KCl 2.67 mM, KH₂PO₄ 1.47 mM, NaCl 137.9 mM, Na₂HPO₄-7H₂O 8.06 mM).

In some embodiments, the solid support is coated with human polyvalent immunoglobulins using a coating solution comprising human polyvalent immunoglobulins at a concentration of at least about 1 μg/mL, 3 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 150 μg/mL, 200 μg/mL, 250 μg/mL, 300 μg/mL, 350 μg/mL, 400 μg/mL, 500 μg/mL, 600 μg/mL, 700 μg/mL, 800 μg/mL, 900 μg/mL, or 1000 μg/mL, preferably at a concentration of at least about 3 μg/mL. Thus, in some embodiments, the coating solution as described above comprises human polyvalent immunoglobulins at a concentration of at least about 1, 3, 10, 20, 30, 40, 50, 60, 70, 80, 90, 100, 150, 200, 250, 300, 350, 400, 500, 600, 700, 800, 900, or 1000 μg/mL, preferably at a concentration of at least about 3 μg/mL.

In some embodiments, the solid support is coated with human polyvalent immunoglobulins using a coating solution comprising human polyvalent immunoglobulins at a concentration ranging from about 1 μg/mL to about 1000 μg/mL, preferably from about 3 μg/mL to about 300 μg/mL, more preferably from about 10 μg/mL to about 100 μg/mL. Thus, in some embodiments, the coating solution as described above comprises human polyvalent immunoglobulins at a concentration ranging from about 1 μg/mL to about 1000 μg/mL, preferably from about 3 μg/mL to about 300 μg/mL, more preferably from about 10 μg/mL to about 100 μg/mL.

In some embodiments, the solid support is coated with human polyvalent immunoglobulins using a coating solution comprising human polyvalent immunoglobulins at a concentration of about 1 μg/mL, 3 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 150 μg/mL, 200 μg/mL, 250 μg/mL, 300 μg/mL, 350 μg/mL, 400 μg/mL, 500 μg/mL, 600 μg/mL, 700 μg/mL, 800 μg/mL, 900 μg/mL, or 1000 μg/mL. Thus, in some embodiments, the coating solution as described above comprises human polyvalent immunoglobulins at a concentration of about 1, 3, 10, 20, 30, 40, 50, 60, 70, 80, 90, 100, 150, 200, 250, 300, 350, 400, 500, 600, 700, 800, 900, or 1000 μg/mL.

In some embodiments, ET formation and/or ETosis is detected using a cell-impermeant nucleic acid dye. In some embodiments, NET formation and/or NETosis is detected using a cell-impermeant nucleic acid dye.

Accordingly, in some embodiments, the cells, preferably neutrophils, are cultured in a culture medium comprising a cell-impermeant nucleic acid dye. In some embodiments, the biological sample comprising the cells, preferably neutrophils, is diluted in a culture medium comprising a cell-impermeant nucleic acid dye. In some embodiments, the cell-impermeant nucleic acid dye is fluorescent. Examples of fluorescent cell-impermeant nucleic acid dyes suitable for cell culture include Sytox Green.

In some embodiments, the method comprises assessing ET formation, preferably NET formation, and/or ETosis, preferably NETosis, by image analysis. In some embodiments, the method comprises assessing ET formation, preferably NET formation, and/or ETosis, preferably NETosis, by live image analysis. In some embodiments, the method comprises assessing ET formation, preferably NET formation, and/or ETosis, preferably NETosis, by continuous image acquisition. In some embodiments, the method comprises assessing ET formation, preferably NET formation, and/or ETosis, preferably NETosis, by image acquisition at specific time points.

In some embodiments, the method comprises assessing ET formation, preferably NET formation, and/or ETosis, preferably NETosis, by fluorescence detection. Methods for detecting fluorescence, in particular for quantitatively detecting fluorescence, are well-known to one skilled in the art. Examples of such methods include using a fluorometer, a plate reader or any other apparatus able to detect and quantify fluorescent light signals.

In some embodiments, the method comprises assessing ET formation, preferably NET formation, and/or ETosis, preferably NETosis, by fluorescent microscopy. In some embodiments, the method comprises assessing ET formation, preferably NET formation, and/or ETosis, preferably NETosis, by live cell fluorescent microscopy.

In some embodiments, the method comprises quantifying ET formation, preferably NET formation, and/or ETosis, preferably NETosis. In some embodiments, the method comprises quantifying ET formation, preferably NET formation, and/or ETosis, preferably NETosis, by computer-assisted image analysis.

In some embodiments, ET formation, preferably NET formation, can be detected after 1 hour, 2 hours, 3 hours, 4 hours, 5 hours, 6 hours, 7 hours, 8 hours, 9 hours, or hours of culture of the cells, preferably neutrophils, in contact with human polyvalent immunoglobulins immobilized on a solid support. In some embodiments, ET formation, preferably NET formation, can be detected after 1 hour, 2 hours, 3 hours, 4 hours, 5 hours, 6 hours, 7 hours, 8 hours, 9 hours, or 10 hours of contact of the biological sample comprising the cells, preferably neutrophils, with human polyvalent immunoglobulins immobilized on a solid support.

Another object of the present invention is a kit comprising:
a solid support as described above;
a coating solution as described above;
human polyvalent immunoglobulins as described above; and optionally
instructions for use.

In some embodiments, implementing the kit allows to obtain a solid support coated with human polyvalent immunoglobulins, that is to say human polyvalent immunoglobulins immobilized on a solid support.

Another object of the present invention is a kit comprising:
a solid support as described above;
a coating solution comprising soluble human polyvalent immunoglobulins as described above; and optionally
instructions for use.
   In some embodiments, implementing the kit allows to obtain a solid support coated with human polyvalent immunoglobulins, that is to say human polyvalent immunoglobulins immobilized on a solid support.

By “kit” is intended any manufacture (e.g., a package or a container) comprising a solid support, a coating solution, human polyvalent immunoglobulins, and optionally instructions for use. The kit may be promoted, distributed, or sold as a unit for performing the methods described herein.

Another object of the present invention is an in vitro method for screening a drug for its ability to modulate extracellular trap (ET) formation and/or ETosis, in particular neutrophil extracellular trap (NET) formation and/or NETosis comprising:
a) contacting immune cells, in particular neutrophils in in vitro culture with a drug;
b) inducing ET formation and/or ETosis, in particular NET formation and/or NETosis by contacting the immune cells, in particular neutrophils in in vitro culture with human polyvalent immunoglobulins immobilized on a solid support as described above; and
c) assessing the effect of the drug on ET formation and/or ETosis, in particular NET formation and/or NETosis in the in vitro culture of immune cells, in particular neutrophils, wherein the order of step a) and b) can be inverted.

In some embodiments, the method comprises:
a) contacting immune cells, in particular neutrophils in in vitro culture with a drug;
b) inducing ET formation and/or ETosis, in particular NET formation and/or NETosis by contacting the immune cells, in particular neutrophils in in vitro culture with human polyvalent immunoglobulins immobilized on a solid support as described above; and
c) assessing the effect of the drug on ET formation and/or ETosis, in particular NET formation and/or NETosis in the in vitro culture of immune cells, in particular neutrophils.

In some embodiments, the method comprises:
a) inducing ET formation and/or ETosis, in particular NET formation and/or NETosis by contacting immune cells, in particular neutrophils in in vitro culture with human polyvalent immunoglobulins immobilized on a solid support as described above;
b) contacting the immune cells, in particular neutrophils in in vitro culture with a drug; and
c) assessing the effect of the drug on ET formation and/or ETosis, in particular NET formation and/or NETosis in the in vitro culture of immune cells, in particular neutrophils.

In some embodiments, rather than being in culture, the immune cells, in particular neutrophils, may be comprised within a biological sample, such as a blood sample.

In some embodiments, by “contacting immune cells, in particular neutrophils in in vitro culture with a drug” it is meant adding a drug to the culture medium in which the immune cells, in particular neutrophils are cultured. In some embodiments, when the immune cells, in particular neutrophils, are comprised within a biological sample, by “contacting with a drug”, it is meant adding a drug to the biological sample or to the culture medium in which the biological sample is diluted.

In some embodiments, the method for screening a drug for its ability to modulate ET formation and/or ETosis, in particular NET formation and/or NETosis further comprises comparing the ET formation and/or ETosis, in particular NET formation and/or NETosis obtained in presence of the drug to a reference.

In some embodiments, the reference is the ET formation and/or ETosis, in particular NET formation and/or NETosis obtained in the absence of the drug. In some embodiments, the reference is the ET formation and/or ETosis, in particular NET formation and/or NETosis obtained in the presence of a drug known to inhibit or decrease ET formation and/or ETosis, in particular NET formation and/or NETosis. Examples of drugs known to inhibit or decrease ET formation and/or ETosis, in particular NET formation and/or NETosis include disulfiram; peptidyl arginine deiminase 4 (PAD4) inhibitors, such as Cl-amidine; neutrophil elastase (NE) inhibitors, such as sivelestat (also known as elaspol), alvelestat; DNases, such as DNase I; and anti-citrullinated protein antibodies (ACP) also known as therapeutic anti-citrullinated protein antibody (tACPA), such as CIT-013. In some embodiments, the reference is the ET formation and/or ETosis, in particular NET formation and/or NETosis obtained in the presence of a drug known to stimulate or increase ET formation and/or ETosis, in particular NET formation and/or NETosis. Examples of drugs known to stimulate or increase ET formation and/or ETosis, in particular NET formation and/or NETosis include sulfasalazine (SSZ).

Another object of the present invention is an in vitro method for assessing the extracellular trap (ET) formation and/or ETosis, in particular neutrophil extracellular trap (NET) formation and/or NETosis, in a subject comprising:
a) contacting a biological sample comprising immune cells, in particular neutrophils, or an in vitro culture of immune cells, in particular neutrophils, previously obtained from a subject, with human polyvalent immunoglobulins immobilized on a solid support, thereby inducing ET formation and/or ETosis, in particular NET formation and/or NETosis;
b) detecting and/or quantifying ET formation and/or ETosis, in particular NET formation and/or NETosis induced in the biological sample or in the in vitro culture of immune cells, in particular neutrophils.

Another object of the present invention is an in vitro method for assessing the susceptibility of a subject to extracellular trap (ET) formation and/or ETosis, in particular neutrophil extracellular trap (NET) formation and/or NETosis comprising:
a) contacting a biological sample comprising immune cells, in particular neutrophils, or an in vitro culture of immune cells, in particular neutrophils, previously obtained from a subject, with human polyvalent immunoglobulins immobilized on a solid support, thereby inducing ET formation and/or ETosis, in particular NET formation and/or NETosis;
b) detecting and/or quantifying ET formation and/or ETosis, in particular NET formation and/or NETosis induced in the biological sample or in the in vitro culture of immune cells, in particular neutrophils, thereby assessing the susceptibility of the subject to ET formation and/or ETosis, in particular NET formation and/or NETosis.

In some embodiments, the method further comprises a first step of isolating immune cells, in particular neutrophils from a biological sample, such as a peripheral blood sample, previously obtained from a subject. As indicated above, methods for isolating immune cells, in particular neutrophils from a blood sample are commonly known. However, in some embodiments, the method does not comprise an active step of recovering a biological sample from the subject. Consequently, according to some embodiments, the method is a non-invasive method, i.e., the method is an in vitro method.

In some embodiments, the method further comprises determining the minimal ratio of immune cells:human polyvalent immunoglobulins, in particular neutrophils:human polyvalent immunoglobulins required to induce ET formation and/or ETosis, in particular NET formation and/or NETosis in the biological sample or in the in vitro culture of immune cells, in particular neutrophils previously obtained from a subject. For example, the minimal ratio of immune cells:human polyvalent immunoglobulins, in particular neutrophils:human polyvalent immunoglobulins required to induce ET formation and/or ETosis, in particular NET formation and/or NETosis may be determined by contacting in vitro cultures of a given concentration (e.g., about 1×10⁶ cells/mL of culture medium) of immune cells, in particular neutrophils, previously obtained from a subject, with increasing densities of human polyvalent immunoglobulins immobilized on a solid support.

In some embodiments, the method further comprises determining the ratio of immune cells:human polyvalent immunoglobulins, in particular neutrophils:human polyvalent immunoglobulins required to induce maximal ET formation and/or ETosis, in particular NET formation and/or NETosis in the biological sample or in the in vitro culture of immune cells, in particular neutrophils, previously obtained from a subject. For example, the ratio of immune cells:human polyvalent immunoglobulins, in particular neutrophils:human polyvalent immunoglobulins required to induce maximal ET formation and/or ETosis, in particular NET formation and/or NETosis may be determined by contacting in vitro cultures of a given concentration (e.g., about 1×10⁶ cells/mL of culture medium) of immune cells, in particular neutrophils, previously obtained from a subject, with increasing densities of human polyvalent immunoglobulins immobilized on a solid support.

In some embodiments, the susceptibility of a subject to ET formation and/or ETosis, in particular NET formation and/or NETosis is determined based on the minimal ratio of immune cells:human polyvalent immunoglobulins, in particular neutrophils:human polyvalent immunoglobulins required to induce ET formation and/or ETosis, in particular NET formation and/or NETosis in the in vitro culture of immune cells, in particular neutrophils, previously obtained from a subject.

In some embodiments, the susceptibility of a subject to ET formation and/or ETosis, in particular NET formation and/or NETosis is determined based on the ratio of immune cells:human polyvalent immunoglobulins, in particular neutrophils:human polyvalent immunoglobulins required to induce maximal ET formation and/or ETosis, in particular NET formation and/or NETosis in the in vitro culture of immune cells, in particular neutrophils, previously obtained from a subject.

For example, in some embodiments, it is considered that:
when the ratio of immune cells:human polyvalent immunoglobulins, in particular neutrophils:human polyvalent immunoglobulins required to induce maximal ET formation and/or ETosis, in particular NET formation and/or NETosis in the in vitro culture of immune cells, in particular neutrophils, previously obtained from a subject, is a high ratio of immune cells:human polyvalent immunoglobulins, in particular neutrophils:human polyvalent immunoglobulins, the subject is a high responder (that is to say the subject is very susceptible or shows high susceptibility to ET formation and/or ETosis, in particular NET formation and/or NETosis);
when the ratio of immune cells:human polyvalent immunoglobulins, in particular neutrophils:human polyvalent immunoglobulins required to induce maximal ET formation and/or ETosis, in particular NET formation and/or NETosis in the in vitro culture of immune cells, in particular neutrophils, previously obtained from a subject, is an intermediate ratio of immune cells:human polyvalent immunoglobulins, in particular neutrophils:human polyvalent immunoglobulins, the subject is an intermediate responder (that is to say the subject is susceptible or shows susceptibility to ET formation and/or ETosis, in particular NET formation and/or NETosis); and/or
when the ratio of immune cells:human polyvalent immunoglobulins, in particular neutrophils:human polyvalent immunoglobulins required to induce maximal ET formation and/or ETosis, in particular NET formation and/or NETosis in the in vitro culture of immune cells, in particular neutrophils, previously obtained from a subject, is a low ratio of immune cells:human polyvalent immunoglobulins, in particular neutrophils:human polyvalent immunoglobulins, the subject is a low responder (that is to say the subject is not very susceptible or shows little susceptibility to ET formation and/or ETosis, in particular NET formation and/or NETosis).

In some embodiments, a high responder (that is to say a subject very susceptible or showing high susceptibility to ET formation and/or ETosis, in particular NET formation and/or NETosis) corresponds to a subject showing maximal ET formation and/or ETosis, in particular NET formation and/or NETosis at a low concentration or density of immobilized human polyvalent immunoglobulins and thus at a high ratio of immune cells:human polyvalent immunoglobulins, in particular neutrophils:human polyvalent immunoglobulins.

In some embodiments, a high ratio of immune cells:human polyvalent immunoglobulins, in particular neutrophils:human polyvalent immunoglobulins corresponds:
to a culture of immune cells, in particular neutrophils at a concentration ranging from about 1×10⁵ to about 1×10⁷ cells/mL of culture medium, preferably from about 5×10⁵ to about 5×10⁶ cells/mL of culture medium, more preferably at a concentration of about 1×10⁶ cells/mL of culture medium; and
to a solid support coated with a coating solution comprising human polyvalent immunoglobulins at a concentration ranging from about 1 μg/mL to about 20 μg/mL, preferably from about 3 μg/mL to about 15 μg/mL, more preferably at a concentration of about 10 μg/mL.

In some embodiments, an intermediate responder (that is to say a subject susceptible or showing susceptibility to ET formation and/or ETosis, in particular NET formation and/or NETosis) corresponds to a subject showing maximal ET formation and/or ETosis, in particular NET formation and/or NETosis at an intermediate concentration or density of immobilized human polyvalent immunoglobulins and thus at an intermediate ratio of immune cells:human polyvalent immunoglobulins, in particular neutrophils:human polyvalent immunoglobulins.

In some embodiments, an intermediate ratio of immune cells:human polyvalent immunoglobulins, in particular neutrophils:human polyvalent immunoglobulins corresponds for example:
to a culture of immune cells, in particular neutrophils at a concentration ranging from about 1×10⁵ to about 1×10⁷ cells/mL of culture medium, preferably from about 5×10⁵ to about 5×10⁶ cells/mL of culture medium, more preferably at a concentration of about 1×10⁶ cells/mL of culture medium; and
to a solid support coated with a coating solution comprising human polyvalent immunoglobulins at a concentration ranging from about 20 μg/mL to about 50 μg/mL, preferably from about 25 μg/mL to about 40 μg/mL, more preferably at a concentration of about 30 μg/mL.

In some embodiments, a low responder (that is to say a subject not very susceptible or showing little susceptibility to ET formation and/or ETosis, in particular NET formation and/or NETosis) corresponds to a subject showing maximal ET formation and/or ETosis, in particular NET formation and/or NETosis at a high concentration or density of immobilized human polyvalent immunoglobulins and thus at low ratio of immune cells:human polyvalent immunoglobulins, in particular neutrophils:human polyvalent immunoglobulins.

In some embodiments, a low ratio of immune cells:human polyvalent immunoglobulins, in particular neutrophils: human polyvalent immunoglobulins corresponds for example:
to a culture of immune cells, in particular neutrophils at a concentration ranging from about 1×10⁵ to about 1×10⁷ cells/mL of culture medium, preferably from about 5×10⁵ to about 5×10⁶ cells/mL of culture medium, more preferably at a concentration of about 1×10⁶ cells/mL of culture medium; and
to a solid support coated with a coating solution comprising human polyvalent immunoglobulins at a concentration ranging from about 50 μg/mL to about 120 μg/mL, preferably from about 75 μg/mL to about 110 μg/mL, more preferably at a concentration of about 100 μg/mL.

Another object of the present invention is a method for treating a subject susceptible to ET formation and/or ETosis, in particular NET formation and/or NETosis, said method comprising:
a) identifying a subject susceptible to ET formation and/or ETosis, in particular NET formation and/or NETosis by contacting a biological sample comprising immune cells, in particular neutrophils, or an in vitro culture of immune cells, in particular neutrophils, previously obtained from the subject, with human polyvalent immunoglobulins immobilized on a solid support, thereby inducing ET formation and/or ETosis, in particular NET formation and/or NETosis, as described above;
b) treating the subject susceptible to ET formation and/or ETosis, in particular NET formation and/or NETosis by administering to the subject an inhibitor of ET formation and/or ETosis, in particular NET formation and/or NETosis.

Examples of inhibitors of ET formation and/or ETosis, in particular NET formation and/or NETosis include disulfiram; peptidyl arginine deiminase 4 (PAD4) inhibitors, such as Cl-amidine; neutrophil elastase (NE) inhibitors, such as sivelestat (also known as elaspol), alvelestat; DNases, such as DNase I; and anti-citrullinated protein antibodies (ACP) also known as therapeutic anti-citrullinated protein antibody (tACPA), such as CIT-013.

In some embodiments, the step of identifying a subject susceptible to ET formation and/or ETosis, in particular NET formation and/or NETosis further comprises determining the ratio of immune cells:human polyvalent immunoglobulins, in particular neutrophils:human polyvalent immunoglobulins required to induce maximal ET formation and/or ETosis, in particular NET formation and/or NETosis in the biological sample or in the in vitro culture of immune cells, in particular neutrophils, previously obtained from the subject. For example, the ratio of immune cells:human polyvalent immunoglobulins, in particular neutrophils:human polyvalent immunoglobulins required to induce maximal ET formation and/or ETosis, in particular NET formation and/or NETosis may be determined by contacting in vitro cultures of a given concentration (e.g., about 1×10⁶ cells/mL of culture medium) of immune cells, in particular neutrophils, previously obtained from the subject, with increasing densities of human polyvalent immunoglobulins immobilized on a solid support.

Indeed, in some embodiments, a subject susceptible to ET formation and/or ETosis, in particular NET formation and/or NETosis is identified based on the ratio of immune cells:human polyvalent immunoglobulins, in particular neutrophils:human polyvalent immunoglobulins required to induce maximal ET formation and/or ETosis, in particular NET formation and/or NETosis in the biological sample or in the in vitro culture of immune cells, in particular neutrophils, previously obtained from the subject.

In some embodiments, a subject susceptible to ET formation and/or ETosis, in particular NET formation and/or NETosis is a subject identified as a high responder or as an intermediate responder, as described above. In some embodiments, a subject susceptible to ET formation and/or ETosis, in particular NET formation and/or NETosis is a subject identified as a high responder as described above.

The present invention further relates to an in vitro method for predicting the response of a subject to a modulator of ET formation and/or ETosis, in particular of NET formation and/or NETosis, comprising:
a) contacting a biological sample comprising immune cells, in particular neutrophils, or an in vitro culture of immune cells, in particular neutrophils, previously obtained from a subject, with a modulator of ET formation and/or ETosis, in particular NET formation and/or NETosis;
b) inducing ET formation and/or ETosis, in particular NET formation and/or NETosis by contacting the biological sample or the in vitro culture of immune cells, in particular neutrophils, with human polyvalent immunoglobulins immobilized on a solid support as described above; and
c) assessing the effect of the modulator of ET formation and/or ETosis, in particular NET formation and/or NETosis on ET formation and/or ETosis, in particular NET formation and/or NETosis in the biological sample or in the in vitro culture of immune cells, in particular neutrophils,
   wherein the order of step a) and b) can be inverted.

In some embodiments, the method comprises:
a) contacting a biological sample comprising immune cells, in particular neutrophils, or an in vitro culture of immune cells, in particular neutrophils, previously obtained from a subject, with a modulator of ET formation and/or ETosis, in particular NET formation and/or NETosis;
b) inducing ET formation and/or ETosis, in particular NET formation and/or NETosis by contacting the biological sample or the in vitro culture of immune cells, in particular neutrophils, with human polyvalent immunoglobulins immobilized on a solid support as described above; and
c) assessing the effect of the modulator of ET formation and/or ETosis, in particular NET formation and/or NETosis on ET formation and/or ETosis, in particular NET formation and/or NETosis in the biological sample or in the in vitro culture of immune cells, in particular neutrophils.

In some embodiments, the method comprises:
a) inducing ET formation and/or ETosis, in particular NET formation and/or NETosis by contacting a biological sample comprising immune cells, in particular neutrophils, or an in vitro culture of immune cells, in particular neutrophils, previously obtained from a subject, with human polyvalent immunoglobulins immobilized on a solid support as described above;
b) contacting the biological sample or the immune cells, in particular neutrophils in in vitro culture with a modulator of ET formation and/or ETosis, in particular NET formation; and/or NETosis and
c) assessing the effect of the modulator of ET formation and/or ETosis, in particular NET formation and/or NETosis on ET formation and/or ETosis, in particular NET formation and/or NETosis in the biological sample or in the in vitro culture of immune cells, in particular neutrophils.

In some embodiments, the modulator of ET formation and/or ETosis, in particular NET formation and/or NETosis is an inhibitor of ET formation and/or ETosis, in particular NET formation and/or NETosis. In some embodiments, the modulator of ET formation and/or ETosis, in particular NET formation and/or NETosis is a stimulator of ET formation and/or ETosis, in particular NET formation and/or NETosis.

In some embodiments, the method comprises quantifying ET formation and/or ETosis, in particular NET formation and/or NETosis induced in the biological sample or in the in vitro culture of immune cells, in particular neutrophils in presence of the modulator of ET formation and/or ETosis, in particular NET formation and/or NETosis and comparing said ET formation and/or ETosis, in particular NET formation and/or NETosis to a reference value, thereby assessing the response of the subject to a modulator of ET formation and/or ETosis, in particular NET formation and/or NETosis.

In some embodiments, the reference value is the ET formation and/or ETosis, in particular NET formation and/or NETosis induced in a biological sample or in an in vitro culture of immune cells, in particular neutrophils, previously obtained from the subject, in the absence of the modulator of ET formation and/or ETosis, in particular NET formation and/or NETosis.

In some embodiments, when the ET formation and/or ETosis, in particular NET formation and/or NETosis induced in the biological sample or in the in vitro culture of immune cells, in particular neutrophils in presence of the modulator of ET formation and/or ETosis, in particular NET formation and/or NETosis is substantially different compared to the reference value, the subject is considered to be a responder (i.e., to respond to the modulator of ET formation and/or ETosis, in particular NET formation and/or NETosis).

In some embodiments, the ET formation and/or ETosis, in particular NET formation and/or NETosis induced in the biological sample or in the in vitro culture of immune cells, in particular neutrophils in presence of the modulator of ET formation and/or ETosis, in particular NET formation and/or NETosis is substantially different if it is more than about 1% higher, 2% higher, 3% higher, 4% higher, 5% higher, 6% higher, 7% higher, 8% higher, 9% higher, 10% higher, 15% higher, 20% higher, 25% higher, 30% higher, 35% higher, 40% higher, 45% higher, or 50% higher, or more than the reference value; or if it is more than about 1% lower, 2% lower, 3% lower, 4% lower, 5% lower, 6% lower, 7% lower, 8% lower, 9% lower, 10% lower, 15% lower, 20% lower, 25% lower, 30% lower, 35% lower, 40% lower, 45% lower, or 50% lower, or more than the reference value. In one embodiment, the ET formation and/or ETosis, in particular NET formation and/or NETosis induced in the biological sample or in the in vitro culture of immune cells, in particular neutrophils in presence of the modulator of ET formation and/or ETosis, in particular NET formation and/or NETosis is substantially different if it is more than about 5% higher or 5% lower than the reference value.

In some embodiments, when the ET formation and/or ETosis, in particular NET formation and/or NETosis induced in the biological sample or in the in vitro culture of immune cells, in particular neutrophils in presence of an inhibitor of ET formation and/or ETosis, in particular NET formation and/or NETosis is substantially lower compared to the reference value, the subject is considered to be a responder (i.e., to respond to the inhibitor of ET formation and/or ETosis, in particular NET formation and/or NETosis). In some embodiments, the ET formation and/or ETosis, in particular NET formation and/or NETosis induced in the biological sample or in the in vitro culture of immune cells, in particular neutrophils in presence of an inhibitor of ET formation and/or ETosis, in particular NET formation and/or NETosis is substantially lower if it is more than about 1% lower, 2% lower, 3% lower, 4% lower, 5% lower, 6% lower, 7% lower, 8% lower, 9% lower, 10% lower, 15% lower, 20% lower, 25% lower, 30% lower, 35% lower, 40% lower, 45% lower, or 50% lower, or more than the reference value. In one embodiment, the ET formation and/or ETosis, in particular NET formation and/or NETosis induced in the biological sample or in the in vitro culture of immune cells, in particular neutrophils in presence of an inhibitor of ET formation and/or ETosis, in particular NET formation and/or NETosis is substantially lower if it is more than about 5% lower than the reference value.

In some embodiments, when the ET formation and/or ETosis, in particular NET formation and/or NETosis induced in the biological sample or in the in vitro culture of immune cells, in particular neutrophils in presence of a stimulator of ET formation and/or ETosis, in particular NET formation and/or NETosis is substantially higher compared to the reference value, the subject is considered to be a responder (i.e., to respond to the stimulator of ET formation and/or ETosis, in particular NET formation and/or NETosis). In some embodiments, the ET formation and/or ETosis, in particular NET formation and/or NETosis induced in the biological sample or in the in vitro culture of immune cells, in particular neutrophils in presence of a stimulator of ET formation and/or ETosis, in particular NET formation and/or NETosis is substantially higher if it is more than about 1% higher, 2% higher, 3% higher, 4% higher, 5% higher, 6% higher, 7% higher, 8% higher, 9% higher, 10% higher, 15% higher, 20% higher, 25% higher, 30% higher, 35% higher, 40% higher, 45% higher, or 50% higher, or more than the reference value. In one embodiment, the ET formation and/or ETosis, in particular NET formation and/or NETosis induced in the biological sample or in the in vitro culture of immune cells, in particular neutrophils in presence of a stimulator of ET formation and/or ETosis, in particular NET formation and/or NETosis is substantially higher if it is more than about 5% higher than the reference value.

Another object of the present invention is a method for treating a subject identified as a responder to a modulator of ET formation and/or ETosis, in particular NET formation and/or NETosis, said method comprising:
a) identifying a subject as a responder to a modulator of ET formation and/or ETosis, in particular NET formation and/or NETosis as described above;
b) treating the subject identified as a responder to the modulator of ET formation and/or ETosis, in particular NET formation and/or NETosis by administering to the subject the modulator of ET formation and/or ETosis, in particular NET formation and/or NETosis.

Thus, in some embodiments, identifying a subject as a responder to a modulator of ET formation and/or ETosis, in particular NET formation and/or NETosis comprises:
i. contacting a biological sample comprising immune cells, in particular neutrophils, or an in vitro culture of immune cells, in particular neutrophils, previously obtained from the subject, with a modulator of ET formation and/or ETosis, in particular NET formation and/or NETosis;
ii. inducing ET formation and/or ETosis, in particular NET formation and/or NETosis by contacting the biological sample or the in vitro culture of immune cells, in particular neutrophils, with human polyvalent immunoglobulins immobilized on a solid support as described above; and
iii. assessing the effect of the modulator of ET formation and/or ETosis, in particular NET formation and/or NETosis on ET formation and/or ETosis, in particular NET formation and/or NETosis in the biological sample or in the in vitro culture of immune cells, in particular neutrophils,
   wherein the order of step i and ii can be inverted.

The stimulator of ET formation and/or ETosis, in particular NET formation and/or NETosis may be selected from disulfiram; peptidyl arginine deiminase 4 (PAD4) inhibitors, such as Cl-amidine; neutrophil elastase (NE) inhibitors, such as sivelestat (also known as elaspol), alvelestat; DNases, such as DNase I; anti-citrullinated protein antibodies (ACP) also known as therapeutic anti-citrullinated protein antibody (tACPA), such as CIT-013; and sulfasalazine (SSZ).

In some embodiments, the method comprises quantifying ET formation and/or ETosis, in particular NET formation and/or NETosis induced in the biological sample or in the in vitro culture of immune cells, in particular neutrophils in presence of the modulator of ET formation and/or ETosis, in particular NET formation and/or NETosis and comparing said ET formation and/or ETosis, in particular NET formation and/or NETosis to a reference value, thereby identifying a subject as a responder to a modulator of ET formation and/or ETosis, in particular NET formation and/or NETosis.

In some embodiments, the reference value is the ET formation and/or ETosis, in particular NET formation and/or NETosis induced in a biological sample or in an in vitro culture of immune cells, in particular neutrophils, previously obtained from the subject, in the absence of the modulator of ET formation and/or ETosis, in particular NET formation and/or NETosis.

In some embodiments, when the ET formation and/or ETosis, in particular NET formation and/or NETosis induced in the biological sample or in the in vitro culture of immune cells, in particular neutrophils in presence of the modulator of ET formation and/or ETosis, in particular NET formation and/or NETosis is substantially different compared to the reference value, the subject is considered to be a responder (i.e., to respond to the modulator of ET formation and/or ETosis, in particular NET formation and/or NETosis).

Another object of the present invention is an in vitro method for monitoring the response of a subject to a therapeutic or prophylactic agent, comprising:
a) contacting a biological sample comprising immune cells, in particular neutrophils, or an in vitro culture of immune cells, in particular neutrophils, previously obtained from a subject, with human polyvalent immunoglobulins immobilized on a solid support, thereby inducing ET formation and/or ETosis, in particular NET formation and/or NETosis as described above, wherein said subject was treated or is being treated with a therapeutic or prophylactic agent;
b) quantifying ET formation and/or ETosis, in particular NET formation and/or NETosis induced in the biological sample or in the in vitro culture of immune cells, in particular neutrophils; and
c) comparing the ET formation and/or ETosis, in particular NET formation and/or NETosis induced in the biological sample or in the in vitro culture of immune cells, in particular neutrophils to a reference value, thereby monitoring the response of the subject to the therapeutic or prophylactic agent.

In some embodiments, by “was treated or is being treated with a therapeutic or prophylactic agent” it is meant that the subject was administered with or is being administered with a therapeutic or prophylactic agent.

In some embodiments, the therapeutic or prophylactic agent is an immunomodulator. Thus, in some embodiments, the present invention relates to an in vitro method for monitoring the response of a subject to an immunomodulator, comprising:
a) contacting a biological sample comprising immune cells, in particular neutrophils, or an in vitro culture of immune cells, in particular neutrophils, previously obtained from a subject, with human polyvalent immunoglobulins immobilized on a solid support, thereby inducing ET formation and/or ETosis, in particular NET formation and/or NETosis as described above, wherein said subject was treated or is being treated with an immunomodulator;
b) quantifying ET formation and/or ETosis, in particular NET formation and/or NETosis induced in the biological sample or in the in vitro culture of immune cells, in particular neutrophils; and
c) comparing the ET formation and/or ETosis, in particular NET formation and/or NETosis induced in the biological sample or in the in vitro culture of immune cells, in particular neutrophils to a reference value, thereby monitoring the response of the subject to the immunomodulator.

In some embodiments, “immunomodulator” refers to a substance, in particular a therapeutic drug, that modulates an immune or inflammatory response by increasing (immunostimulant or immunostimulator) or decreasing (immunosuppressant or immunosuppressor) the immune or inflammatory response. Thus, the immunomodulator may be an immunostimulant (or immunostimulator) or an immunosuppressant (or immunosuppressor). For example, an immunostimulant (or immunostimulator) may increase an immune or inflammatory response by inducing activation or increasing activity of immune cells. Conversely, an immunosuppressant (or immunosuppressor) may decrease (or inhibit) an immune or inflammatory response by preventing or inhibiting activation or decreasing activity of immune cells.

In some embodiments, the reference value is the ET formation and/or ETosis, in particular NET formation and/or NETosis, induced in a biological sample or in an in vitro culture of immune cells, in particular neutrophils, previously obtained from the subject before treatment with the therapeutic or prophylactic agent. Thus, in other words, in some embodiments, the reference value is the ET formation and/or ETosis, in particular NET formation and/or NETosis, induced in a biological sample or in an in vitro culture of immune cells, in particular neutrophils, previously obtained from the subject before said subject was administered with the therapeutic or prophylactic agent.

In some embodiments, when the ET formation and/or ETosis, in particular NET formation and/or NETosis, induced in the biological sample or in the in vitro culture of immune cells, in particular neutrophils, previously obtained from a subject who was treated or is being treated with a therapeutic or prophylactic agent is substantially different compared to the reference value, the subject is considered to respond to the therapeutic or prophylactic agent.

In some embodiments, the ET formation and/or ETosis, in particular NET formation and/or NETosis, induced in the biological sample or in the in vitro culture of immune cells, in particular neutrophils, previously obtained from a subject who was treated or is being treated with a therapeutic or prophylactic agent is substantially different if it is more than about 1% higher, 2% higher, 3% higher, 4% higher, 5% higher, 6% higher, 7% higher, 8% higher, 9% higher, 10% higher, 15% higher, 20% higher, 25% higher, 30% higher, 35% higher, 40% higher, 45% higher, or 50% higher, or more than the reference value; or if it is more than about 1% lower, 2% lower, 3% lower, 4% lower, 5% lower, 6% lower, 7% lower, 8% lower, 9% lower, 10% lower, 15% lower, 20% lower, 25% lower, 30% lower, 35% lower, 40% lower, 45% lower, or 50% lower, or more than the reference value. In some embodiments, the ET formation and/or ETosis, in particular NET formation and/or NETosis, induced in the biological sample or in the in vitro culture of immune cells, in particular neutrophils, previously obtained from a subject who was treated or is being treated with a therapeutic or prophylactic agent is substantially different if it is more than about 5% higher or 5% lower than the reference value.

In some embodiments, when the ET formation and/or ETosis, in particular NET formation and/or NETosis, induced in the biological sample or in the in vitro culture of immune cells, in particular neutrophils, previously obtained from a subject who was treated or is being treated with an immunostimulant (or immunostimulator) is substantially higher compared to the reference value, the subject is considered to respond to the immunostimulant (or immunostimulator).

In some embodiments, the ET formation and/or ETosis, in particular NET formation and/or NETosis, induced in the biological sample or in the in vitro culture of immune cells, in particular neutrophils, previously obtained from a subject who was treated or is being treated with an immunostimulant (or immunostimulator) is substantially higher if it is more than about 1% higher, 2% higher, 3% higher, 4% higher, 5% higher, 6% higher, 7% higher, 8% higher, 9% higher, 10% higher, 15% higher, 20% higher, 25% higher, 30% higher, 35% higher, 40% higher, 45% higher, or 50% higher, or more than the reference value. In one embodiment, the ET formation and/or ETosis, in particular NET formation and/or NETosis, induced in the biological sample or in the in vitro culture of immune cells, in particular neutrophils, previously obtained from a subject who was treated or is being treated with an immunostimulant (or immunostimulator) is substantially higher if it is more than about 5% higher than the reference value.

In some embodiments, when the ET formation and/or ETosis, in particular NET formation and/or NETosis, induced in the biological sample or in the in vitro culture of immune cells, in particular neutrophils, previously obtained from a subject who was treated or is being treated with an immunosuppressant (immunosuppressor) is substantially lower compared to the reference value, the subject is considered to respond to the immunosuppressant or (immunosuppressor).

In some embodiments, the ET formation and/or ETosis, in particular NET formation and/or NETosis, induced in the biological sample or in the in vitro culture of immune cells, in particular neutrophils, previously obtained from a subject who was treated or is being treated with an immunosuppressant (immunosuppressor) is substantially lower if it is more than about 1% lower, 2% lower, 3% lower, 4% lower, 5% lower, 6% lower, 7% lower, 8% lower, 9% lower, 10% lower, 15% lower, 20% lower, 25% lower, 30% lower, 35% lower, 40% lower, 45% lower, or 50% lower, or more than the reference value. In one embodiment, the ET formation and/or ETosis, in particular NET formation and/or NETosis, induced in the biological sample or in the in vitro culture of immune cells, in particular neutrophils, previously obtained from a subject who was treated or is being treated with an immunosuppressant (immunosuppressor) is substantially lower if it is more than about 5% lower than the reference value.

In some embodiments, the subject is suffering from a disease selected from an inflammatory disease, a thromboembolic disease, a cancer (such as a solid cancer, a blood cancer, or a lymphoma) and a fibrosis. In some embodiments, said disease is a neutrophil-associated disease or condition. In some embodiments, the neutrophil-associated disease or condition is a disease or condition to which NET formation and/or NETosis may contribute.

Thus, in some embodiments, the neutrophil-associated disease or condition, in particular a disease or condition to which NET formation and/or NETosis may contribute, is an inflammatory disease, a thromboembolic disease, a cancer (such as a solid cancer, a blood cancer, or a lymphoma), or a fibrosis.

In some embodiments, the inflammatory disease is an autoinflammatory disease or an autoimmune disease. In some embodiments, the inflammatory disease is a chronic inflammatory disease. In some embodiments, the inflammatory disease is an acute inflammatory disease.

In some embodiments, the inflammatory disease is vasculitis. In some embodiments, the inflammatory disease is anti-neutrophil cytoplasmic autoantibody (ANCA)-associated vasculitis or ANCA vasculitis.

In some embodiments, the cancer is a solid cancer. Examples of solid cancers include breast cancer, lung cancer, pancreatic cancer, colon cancer, kidney cancer, prostate cancer.

In some embodiments, the cancer is a blood cancer. Examples of blood cancers include acute or chronic myeloid leukemia, acute lymphoblastic leukemia, chronic lymphocytic leukemia, myeloproliferative diseases, multiple myeloma, myelodysplastic syndrome.

In some embodiments, the cancer is a lymphoma. Examples of lymphoma include Hodgkin lymphoma and non-Hodgkin lymphoma (NHL).

In some embodiments, the fibrosis is cystic fibrosis. In some embodiments, the fibrosis is a fibrosis associated with a chronic inflammatory disease or a complication of a chronic inflammatory disease. Examples of such fibroses include liver fibrosis, lung fibrosis, kidney fibrosis or renal fibrosis in chronic kidney disease.

In some embodiments, the neutrophils-associated disease or condition, in particular a disease or condition to which NET formation and/or NETosis may contribute, is selected from the group comprising or consisting of vasculitis, in particular ANCA vasculitis; asthma; rheumatoid arthritis (RA); chronic obstructive pulmonary disease (COPD); systemic lupus erythematous (SLE); systemic sclerosis; adverse cardiovascular events such as myocardial infarction; ischemic stroke; atherosclerosis; venous thrombosis, cancer, in particular solid cancer; fibrosis, for example cystic fibrosis; sepsis; gout; and Alzheimer's disease.

### EXAMPLES

The present invention is further illustrated by the following examples.

### Example 1: Evaluation of NET Formation by Human Neutrophils Interacting with Plate-Bound Human Polyvalent Immunoglobulins

### Materials and Methods

### Reagents

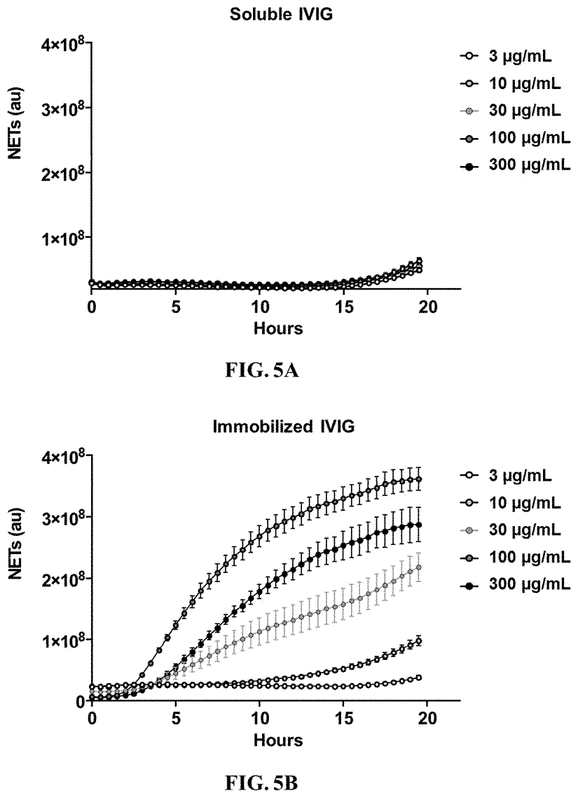

Human polyvalent immunoglobulins (or human polyvalent Ig) also referred to as intravenous immunoglobulin or IVIg (Privigen, CSL Behring LLC) or human serum albumin or HSA (Sigma Aldrich—reference: H5667), used as negative control, diluted in coating solution (DPBS Gibco—reference: 14190) were allowed to adsorb for 2 hours at room temperature on high-binding, sterile 96-well plates suitable for fluorescence microscopy (Greiner Bio-One, Frickenhausen, Germany). Unless indicated otherwise, deposits of human polyvalent Ig were obtained with a coating solution (DPBS no calcium, no magnesium (ThermoFisher—reference: 14190) consisting of KCl 2.67 mM, KH₂PO₄ 1.47 mM, NaCl 137.9 mM, Na₂HPO₄-7H₂O 8.06 mM) comprising human polyvalent Ig at a concentration of 100 μg/mL. Similarly, unless indicated otherwise, deposits of HSA were obtained with a coating solution comprising HSA at a concentration of 100 μg/mL. Phorbol-12-myristate-13-acetate or PMA (Sigma Aldrich—reference: P8139) was used as positive control. PMA was added to the culture medium at a final concentration of nM. In some experiments (see FIG. 5A), human polyvalent Ig were added in the culture medium at a final concentration ranging from 3 to 300 μg/mL.

### Cells and Cell Culture

Neutrophils were isolated from the peripheral blood of healthy human donors by negative magnetic bead selection using a commercially available kit (STEMCELL Technologies—reference: 19666). Isolated neutrophils were then distributed in 96-well plates (Greiner Bio-One, Frickenhausen, Germany), in particular in 96-well plates coated with human polyvalent Ig or HSA as described above, at a concentration of 7.5×10⁴ cells per well, corresponding to 10⁶ cells/mL of culture medium. Neutrophils were then cultured in HBSS with calcium and magnesium (Gibco—reference: 24020-091) under standard conditions (i.e., humidified incubator 37° C. and 5% CO₂).

### NET Detection: Fixed Neutrophils

After 4 hours of culture, neutrophils were fixed with paraformaldehyde or PFA (2%), permeabilized with Triton X100 (0.1%) and stained with 1 μM Sytox Green (Invitrogen—reference: 57020), which specifically stains nucleic acids.

Images were acquired by fluorescence microscopy in the green channel. NET induction was identified by the nuclear morphology of the visualized neutrophils.

### NET Detection: Live Imaging

Freshly isolated human neutrophils were distributed in 96-well plates coated with human polyvalent Ig (obtained with a coating solution comprising human polyvalent Ig at a concentration of 100 μg/mL) or HSA (obtained with a coating solution comprising HSA at a concentration of 100 μg/mL) and cultured for 24 hours in culture medium containing the fluorescent, cell-impermeant, nucleic acid dye Sytox Green (1 μM). Alternatively, as a positive control, freshly isolated human neutrophils were cultured in a 96-well plate in culture medium comprising PMA (25 nM) and Sytox Green (1 μM).

Time-lapse images were acquired by live cell fluorescent microscopy in 9 spots from each well (conditions are run in triplicates). Time-course quantitative measure of NET formation was performed by computer-assisted image analysis. data are expressed as integrated fluorescence intensity (GCU)/μ²/image/well.

### Results

NET Formation is Induced In Vitro by Human Blood Neutrophils Interacting with Plate-Bound Deposits of Human Polyvalent Ig

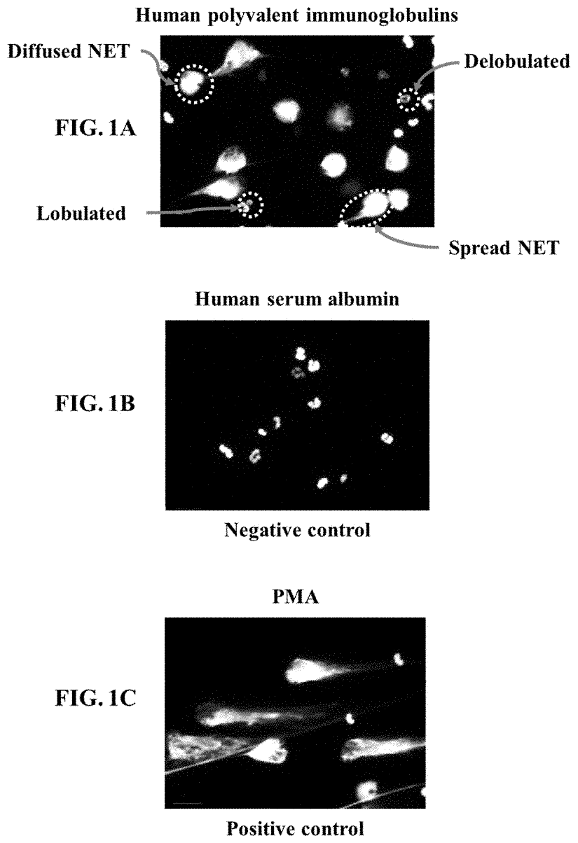

Formation of NETs was assessed in 3 conditions: neutrophils cultured in the presence of plate-bound human polyvalent immunoglobulins (IVIG), neutrophils cultured in the presence of plate-bound HSA (negative control), and neutrophils cultured in the presence of PMA, a known inducer of NETs. Different morphologies of neutrophils undergoing NETosis were observed in wells coated with human polyvalent immunoglobulins: neutrophils with a lobulated nucleus (absence of NET formation), neutrophils with a delobulated nucleus, as well as diffused NETs and spread NETs (FIG. 1A). By contrast, neutrophils all appeared lobulated (absence of NET formation) in the negative control wells coated with human serum albumin (FIG. 1B). In positive control wells (presence of PMA), most neutrophils displayed signs of NETosis, i.e., diffused and spread NETs (FIG. 1C).

These results demonstrate the ability of plate-bound human polyvalent Ig to induce NET formation from isolated human neutrophils.

NET Formation Induced In Vitro by the Interaction of Neutrophils with Immobilized Human Polyvalent Ig is Slow and Progressive

To better characterize the formation of NETs, live imaging was carried out. Three conditions were assessed: neutrophils cultured in the presence of plate-bound human polyvalent Ig (IVIg), neutrophils cultured in the presence of plate-bound HSA (negative control), and neutrophils cultured in the presence of PMA, a known inducer of NETs.

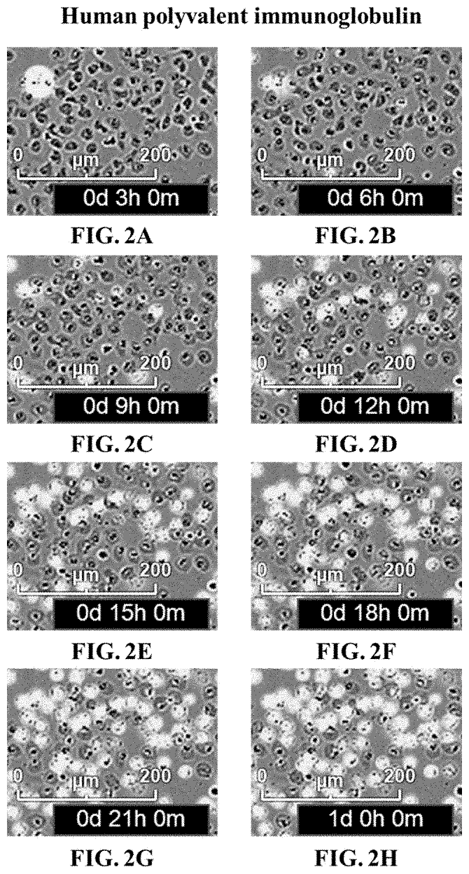

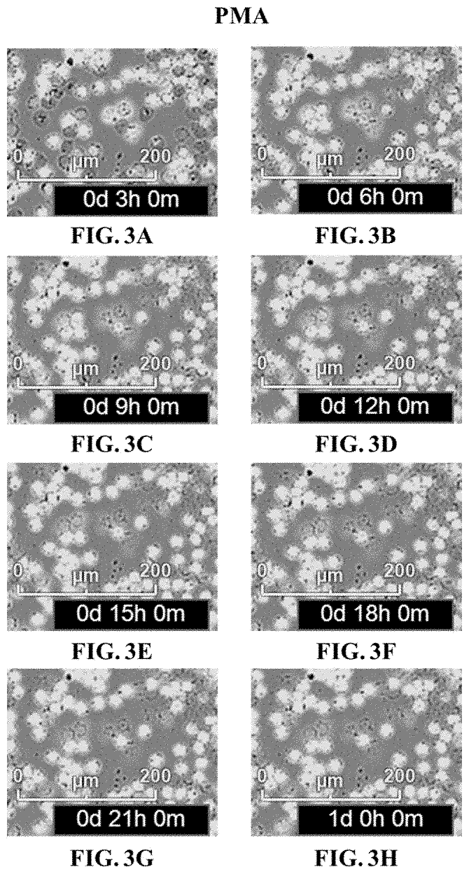

As shown on FIGS. 2A-H, in the presence of plate-bound human polyvalent Ig, progressive NET induction was visible by the immediate staining of extruded nucleic acid, appearing as large fluorescent spots (diffused NETs) at sites of individual neutrophil-Ig interaction at the bottom of the wells (phase contrast) from 3-hour culture to 15-hour culture. By contrast, in the presence of PMA, NET induction was visible on almost all neutrophils from 3-hour culture (see FIG. 3A-H).

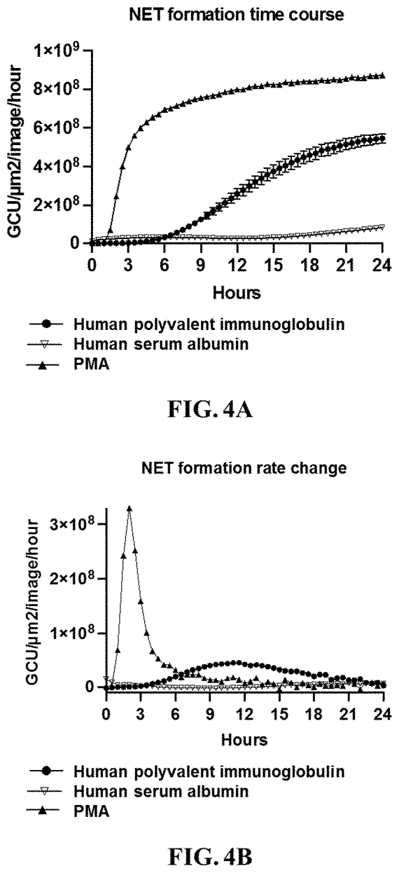

Quantitative assessment of NET formation over time was also performed by computer-assisted image analysis (see FIG. 4). Data reflecting NET formation are expressed as integrated fluorescence intensity (GCU)/μ2/image/well. As shown by the time course of NET formation, the process induced by the interaction of neutrophils with coated human polyvalent Ig is slow and affects discrete cells (FIGS. 2A-H and FIG. 4A), likely reflecting their interaction with immunoglobulin deposits on the bottom of the wells. By contrast, the presence of PMA in the culture medium drives the simultaneous formation of NETs from a great number of neutrophils, within as soon as 3 hours of culture (FIGS. 3A-H and FIG. 4A).

Furthermore, as shown by the NET formation rate change, as opposed to PMA which causes NET formation from all neutrophils at once, coated human polyvalent Ig trigger a progressive and cumulative response (FIG. 4B).

NET Formation is Induced In Vitro by Human Polyvalent Ig Only if the Latter are Immobilized onto a Solid Support

To verify the importance of human polyvalent Ig immobilization, formation of NETs was assessed with neutrophils cultured in the presence of plate-bound human polyvalent Ig and with neutrophils cultured in the presence of human polyvalent Ig added to the culture medium. In both conditions, freshly isolated human peripheral blood neutrophils (7.5×10⁴/well, corresponding to 1×10⁶/mL of culture medium) were cultured in the presence of Sytox Green, with increasing amounts of human polyvalent Ig. Quantitative assessment of NET formation over time was performed by computer-assisted image analysis (arbitrary units, data correspond to integrated green fluorescent intensity expressed as GCU/well).

As shown on FIG. 5A, the use of soluble human polyvalent Ig does not elicit the formation of NETs at any of the tested concentrations. Strikingly, as shown on FIG. 5B, NET formation is clearly elicited by the interaction of neutrophils with immobilized polyvalent human Ig, in a dose-dependent manner. Indeed, the occurrence of NET formation appears to be a function of the ratio neutrophils:immunoglobulins. As shown on FIG. 5B, NET formation starts earlier and increases progressively from 3 μg/mL and up to 100 μg/mL of human polyvalent immunoglobulins (concentration in the coating solution used to prepare the coated culture plates). Of note, NET formation with a concentration of 300 μg/mL of human polyvalent Ig (concentration in the coating solution used to prepare the coated culture plates) starts later and reaches a lower maximal level as compared to the maximal response obtained with a concentration of 100 μg/mL of human polyvalent Ig.

### Example 2: Formation of NETs Induced by Human Polyvalent Immunoglobulins Faithfully Mimics In Vivo SYK-Dependent NET Formation

### Materials and Methods

### Reagents

High-binding, sterile 96-well plates (Greiner Bio-One, Frickenhausen, Germany) were coated with human polyvalent Ig as described in Example 1, using a coating solution comprising human polyvalent Ig at a concentration ranging from 3 to 300 μg/mL, as indicated. If no indication is given, deposits of human polyvalent Ig were obtained with a coating solution comprising human polyvalent Ig at a concentration of 100 μg/mL.

Where indicated, the SYK inhibitor BAY-61-3606, obtained from Selleckchem (reference: S7006), was added to the culture medium at a final concentration ranging from 0.05 to 5 (0.05, 0.1, 0.5, 1, 5 as indicated). Where indicated, the calcineurin inhibitor FK506 (also known as tacrolimus), obtained from ASTELLAS PHARMA (PROGRAF™ 5 mg/ml), was added to the culture medium at a final concentration ranging from 0.3 to 100 nM (0.3, 1, 3, 10, 30, 100 nM, as indicated). Where indicated, the humanized monoclonal antibody motavizumab directed against the respiratory syncytial virus (RSV), produced as a human IgG4 (by Evitria, Zurich, Switzerland), was added to the culture medium at a final concentration ranging from 1 to 100 μg/mL (1, 3, 10, 30, 100 μg/mL, as indicated). Human IgG4 can engage FcRI (CD64) and enhance the activation of human neutrophils triggered by immobilized immunoglobulins, which is mediated by FcRIIa and FcRIIIb, by the engagement of an additional signaling pathway.

### Cells and Cell Culture

Human peripheral blood neutrophils were isolated and cultured as described in Example 1. In particular, freshly isolated peripheral blood neutrophils were distributed in coated plate as described in Example 1 (7.5×10⁴ cells per well, corresponding to 10⁶ cells/mL of culture medium) and cultured for the indicated time.

### Detection

The fluorescent, cell-impermeant, nucleic acid dye Sytox Green was added to the culture medium at a concentration of 1 μM to detect NET formation. ROS (reactive oxygen species) formation was identified by the use of a fluorescent oxidative-sensitive probe (CM-H₂DCFDA, ThermoFisher Scientific—reference: C6827). Cells were pre-incubated for 30 minutes at 37° C. with 5 μM CM-H₂DCFDA in HBSS no calcium, no magnesium. Excess CM-H₂DCFDA was eliminated by cell centrifugation. The labeled cell pellet was resuspended in the culture medium (HBSS with calcium and magnesium).

Time-lapse images were acquired by live cell fluorescent microscopy. Quantitative time-course measure of ROS and NET formation was performed by computer-assisted image analysis (data are expressed as GCU/well).

### Results

### NET Formation Induced In Vitro by Human Polyvalent Ig is Preceded by ROS Formation and Depends on the Density of the Human Polyvalent Ig Deposits

To characterize the underlying mechanism and signaling pathway responsible for the formation of NETs when neutrophils are cultured with plate-bound human polyvalent Ig, the formation of ROS was detected over time, in parallel with the formation of NETs.

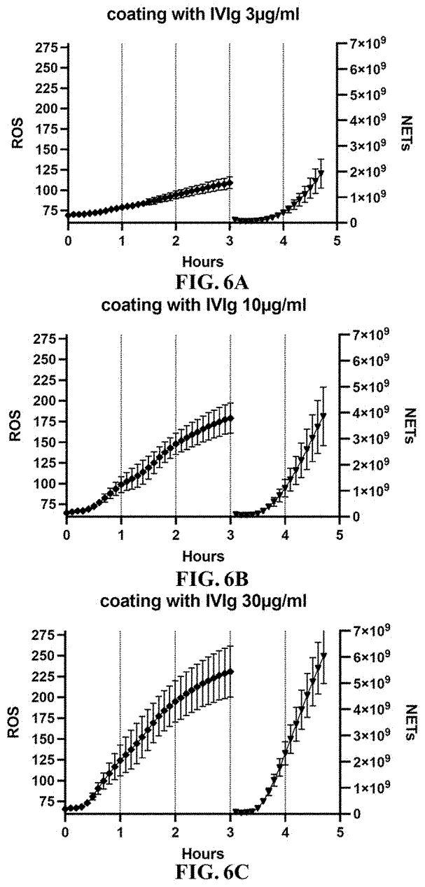

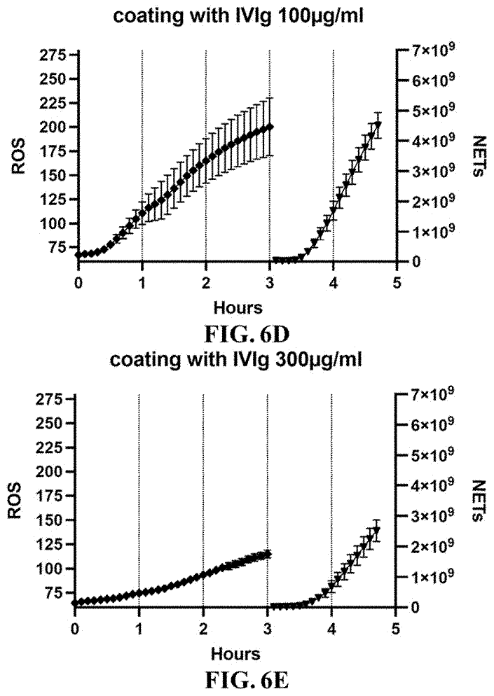

FIGS. 6A-E show the formation of ROS over a 3-hour period starting from the beginning of the culture of neutrophils in a 96-well plate coated with human polyvalent immunoglobulins, and the production of NETs over a 3-hour period starting 3 hours after the beginning of the culture of neutrophils. Several densities of deposits of human polyvalent immunoglobulins were used (obtained with a coating solution comprising human polyvalent immunoglobulins at a concentration ranging from 3 to 300 μg/mL). As shown on the left part of FIGS. 6A-E, ROS formation is rapidly induced, with deposits of human polyvalent immunoglobulins obtained with a coating solution comprising human polyvalent immunoglobulins at a concentration as low as 3 μg/mL (FIG. 6A). ROS formation increases in a dose-dependent manner with deposits of human polyvalent immunoglobulins obtained with a coating solution comprising human polyvalent immunoglobulins at a concentration of 10 μg/mL (FIG. 6B), 30 μg/mL (FIG. 6C), 100 μg/mL (FIG. 6D). ROS formation then decreases when using deposits of human polyvalent immunoglobulins obtained with a coating solution comprising human polyvalent immunoglobulins at a concentration of 300 μg/mL (FIG. 6E). The right part of FIG. 6A-E shows that the formation of NETs occurs after the production of ROS, in a dose dependent-manner. NETs formation increases with deposits of human polyvalent immunoglobulins obtained with a coating solution comprising human polyvalent immunoglobulins at a concentration of 3 μg/mL (FIG. 6A), 10 μg/mL (FIG. 6B), 30 μg/mL (FIG. 6C), and then decreases with deposits of human polyvalent immunoglobulins obtained with a coating solution comprising human polyvalent immunoglobulins at a concentration of 100 μg/mL (FIG. 6D) and 300 μg/mL (FIG. 6E).

These results demonstrate that plate-bound human polyvalent Ig are able to induce NET formation, as well as ROS formation, in a dose-dependent manner. The production of ROS precedes the formation of NETs, as expected given the widely described role of ROS production in NET formation (see for example van der Linden M et al. Sci Rep. 2017 Jul. 26; 7(1):6529).

### NET Formation Induced In Vitro by Human Polyvalent Ig Relies on a SYK Mediated Signaling Pathway

Next, the implication of SYK signaling was investigated by detecting the production of ROS and the formation of NETs over a period of 6 hours (0-360 minutes) when neutrophils are cultured with plate-bound human polyvalent Ig in the absence and in the presence of the SYK inhibitor BAY-61-3606 (also referred herein as SYKi).

The SYK inhibitor BAY-61-3606, obtained from Selleckchem, was thus added at a final concentration ranging from 0 to 5 μM, as indicated, to cultures of freshly isolated human peripheral blood neutrophils in 96-well plates coated with human polyvalent Ig. The coated plates were obtained with a coating solution comprising human polyvalent Ig at a concentration of 100 μg/mL.

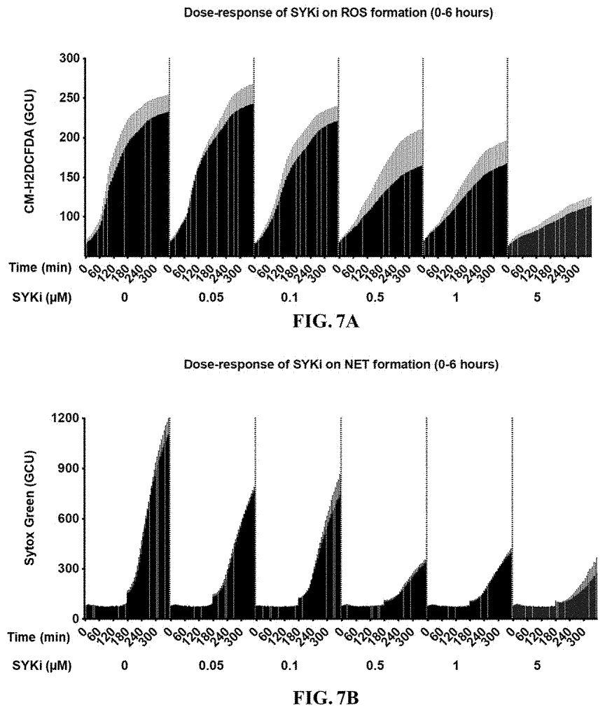

As shown on FIG. 7, both ROS production (FIG. 7A) and NET (FIG. 7B) formation are decreased by the SYK inhibitor BAY-61-3606 in a dose-dependent manner.

These results demonstrate that both ROS production and NET formation induced by immobilized human polyvalent Ig are dependent on the SYK signaling downstream of the Fc receptors expressed at the surface of neutrophils. Moreover, these results demonstrate that immobilized human polyvalent Ig as described herein constitute a physiological inducer of NET formation, which acts via signaling pathways that are involved in the formation of NETs in vivo.

The use of immobilized human polyvalent Ig as described herein thus allows to induce NET formation that is susceptible to be modulated by drugs acting on the signaling pathways that are involved in the formation of NETs in vivo. The use of immobilized human polyvalent Ig as described herein provides a physiologically relevant assay to reproduce NET formation triggered in vivo and can be used to identify, screen and/or assess drugs which may act as immunomodulators, for example to inhibit neutrophil activation and NET formation in the treatment of diseases, such as autoimmune diseases.

### ROS Production Induced In Vitro by Human Polyvalent Ig is Calcium-Dependent

Finally, the importance of calcium signaling was investigated by detecting the production of ROS over a period of 3 hours (0-180 minutes) when neutrophils are cultured with plate-bound human polyvalent Ig in the absence and in the presence of the calcineurin inhibitor FK506 (also known as tacrolimus).

The calcineurin inhibitor FK506 (also known as tacrolimus) was thus added at a final concentration ranging from 0.3 to 100 nM, as indicated, to cultures of freshly isolated human peripheral blood neutrophils in 96-well plates coated with human polyvalent Ig. The coated plates were obtained with a coating solution comprising human polyvalent Ig at a concentration of 100 μg/mL.

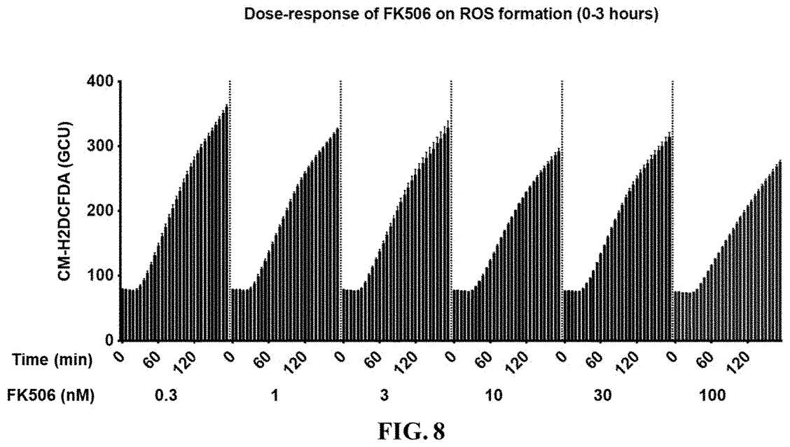

As shown on FIG. 8, ROS production was modestly but consistently reduced in response to the calcineurin inhibitor, in a dose-dependent manner. These results demonstrate that ROS production is blunted, in a dose-dependent way, by the blockade of calcineurin signaling downstream SYK, itself downstream of the Fc receptors expressed at the surface of neutrophils. These results thus confirm that immobilized human polyvalent Ig as described herein constitute a physiological inducer of NET formation, which may be helpful to identify, screen and/or assess drugs targeting the different actors involved in the physiological signaling pathways responsible for NET formation in vivo.

NET Formation Induced In Vitro by Human Polyvalent Ig can be Modulated with Motavizumab, a Monoclonal IgG4

The potentiality for modulation of NET formation was investigated by detecting the formation of NETs over time when neutrophils are cultured with plate-bound human polyvalent Ig in the absence and in the presence of the monoclonal IgG4 motavizumab which can engage FcRI (CD64), thus driving a supplementary SYK-dependent signal, in addition to the one induced by the immobilized human polyvalent Ig (which is mediated by FcRIIa and FcRIIIb).

The monoclonal IgG4 motavizumab was added at a final concentration ranging from 0 to 100 μg/mL, as indicated, to cultures of freshly isolated human peripheral blood neutrophils in 96-well plates coated with human polyvalent Ig. The coated plates were obtained with a coating solution comprising human polyvalent Ig at a concentration of 100 μg/mL. As a control, the SYK inhibitor BAY-61-3606 was used at a concentration of 5 (in the concomitant presence of 100 μg/mL motavizumab and immobilized human polyvalent Ig).

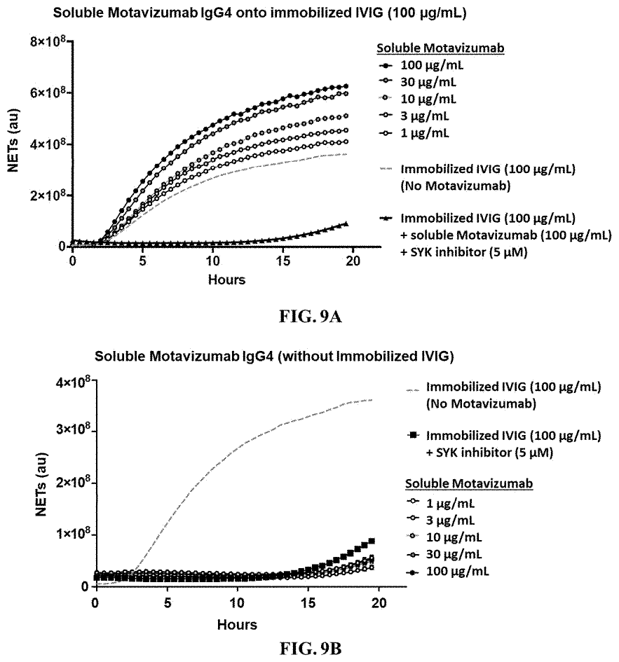

As shown on FIG. 9A, motavizumab added at increasing concentration in the culture medium drives a proportional increase in the formation of NETs as compared to the control (coated IVIG at 100 μg/ml without motavizumab). Of note, the use of the SYK inhibitor BAY-61-3606 completely abolishes the NET formation elicited by the concomitant presence of 100 μg/mL motavizumab and immobilized human polyvalent Ig.

These results thus demonstrate that formation of NETs elicited by immobilized human polyvalent immunoglobulins can be further enhanced by concomitant signaling stimuli.

Next, NET formation was detected after addition of the monoclonal IgG4 motavizumab at a final concentration ranging from 0 to 100 μg/mL, as indicated, to cultures of freshly isolated human peripheral blood neutrophils in uncoated 96-well plates (i.e., in the absence of immobilized human polyvalent Ig). As a positive control, NET formation was detected in freshly isolated human peripheral blood neutrophils cultured in 96-well plates coated with human polyvalent Ig. NET formation was also detected in freshly isolated human peripheral blood neutrophils cultured in the presence of 5 μM of the SYK inhibitor BAY-61-3606 in 96-well plates coated with human polyvalent Ig. The coated plates were obtained with a coating solution comprising human polyvalent Ig at a concentration of 100 μg/mL.

As shown on FIG. 9B, in the absence of immobilized immunoglobulins, the addition of motavizumab at increasing concentration in the culture medium is not able to drive the formation of NETs. By contrast, the formation of NETs is readily detectable when freshly isolated human peripheral blood neutrophils are cultured in 96-well plates coated with human polyvalent Ig. Of note, even when the SYK inhibitor BAY-61-3606 is present at a concentration of 5 μM, the NET formation induced by human polyvalent Ig is greater than the NET formation induced by any of the tested concentrations of motavizumab in the absence of human polyvalent Ig. These results thus demonstrate that motavizumab alone is not able to induce NET formation. Motavizumab acts as an enhancer or amplificator of NET formation induced by human polyvalent Ig. Taken together, the data demonstrate that the use of immobilized human polyvalent Ig as described herein can also allow to identify, screen and/or assess agents or drugs which may act as immunostimulators by enhancing neutrophil activation and NET formation.

### Example 3: Evaluation of NET Formation by Human Neutrophils from Multiple Healthy Donors Induced In Vitro by Plate-Bound Human Polyvalent Ig

### Materials and Methods

### Reagents

High-binding, sterile 96-well plates were coated with human polyvalent Ig as described in Example 1, using a coating solution comprising human polyvalent Ig at a concentration ranging from 3 to 300 μg/mL, as indicated.

### Cells and Cell Culture

Human peripheral blood neutrophils were isolated and cultured as described in Example 1. Blood samples were obtained from 7 healthy human individuals and collected at three independent time points (time points 1, 2, and 3), separated by a least two weeks. Freshly isolated peripheral blood neutrophils were distributed in coated plate as described in Example 1 (7.5×10⁴ cells per well, corresponding to 10⁶ cells/mL of culture medium) and cultured for 16-24 hours in culture medium comprising the fluorescent, cell-impermeant, nucleic acid dye Sytox Green (1 Detection

Time-lapse images were acquired by live cell fluorescent microscopy. Quantitative time-course measure of NET formation was performed by computer-assisted image analysis (data are expressed as area under the curve or AUC).

### Results

### Individuals Display Different Susceptibilities to NET Formation Induced In Vitro by Human Polyvalent Ig

Formation of NETs induced in vitro by plate-bound human polyvalent Ig was assessed using human peripheral blood neutrophils isolated from 7 healthy individuals.

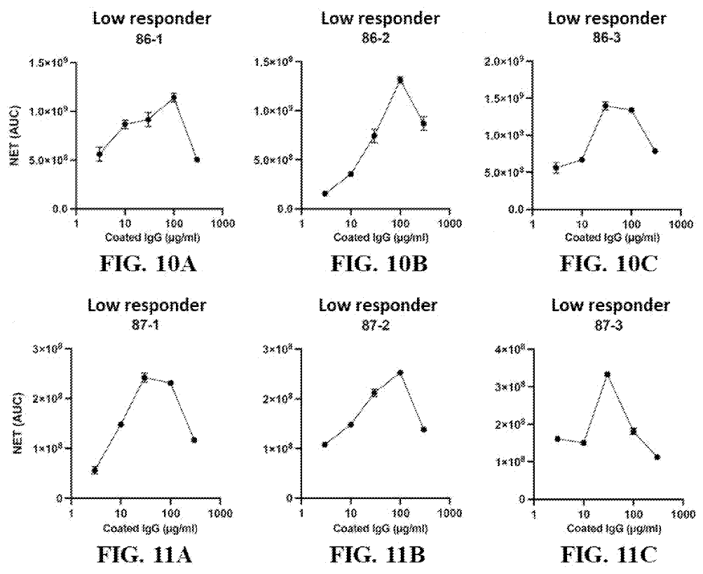

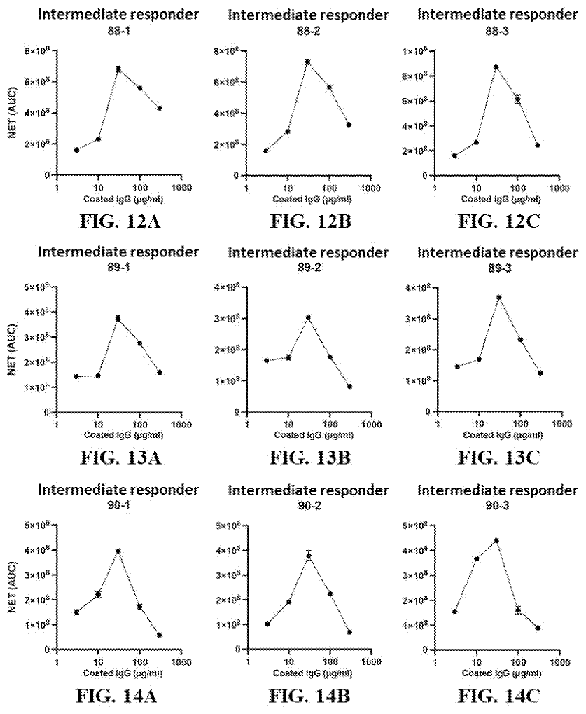

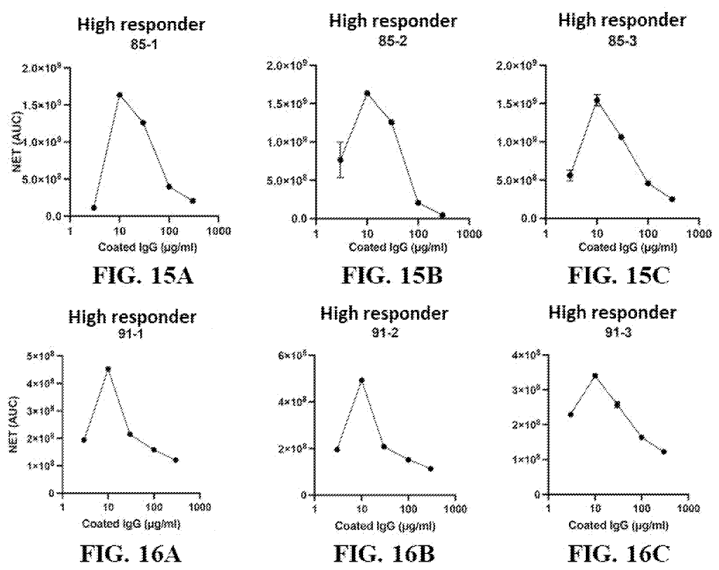

FIGS. 10-16 shows the NET formation induced in vitro by plate-bound human polyvalent Ig with neutrophils isolated from each of the 7 healthy individuals, referred to with a number (individual 86—FIGS. 10A-C, individual 87—FIGS. 11A-C, individual 88—FIGS. 12A-C, individual 89—FIGS. 13A-C, individual 90—FIGS. 14A-C, individual 85—FIGS. 15A-C, and individual 91—FIGS. 16A-C). For each individual, NET formation induced in vitro by plate-bound human polyvalent Ig was assessed using neutrophils isolated from three different blood samples obtained at three independent time points (time points 1—FIGS. 10A-16A, 2—FIGS. 10B-16B, and 3—FIGS. 10C-16C), separated by a least two weeks. For each individual, NET formation was assessed by contacting neutrophils at a fixed concentration of 7.5×10⁴ cells per well, corresponding to 10⁶ cells/mL of culture medium, with increasing densities of plate-bound human polyvalent Ig (obtained with a coating solution comprising human polyvalent Ig at a concentration of 3, 10, 30, 100 or 300 μg/mL).

Strikingly, as shown on FIGS. 10-16, the extent of NET formation observed in response to the engagement of the Fc receptors at the surface of neutrophils by immobilized human polyvalent Ig varies among individuals. Indeed, depending on the individual, maximal NET formation was obtained with differing densities of plate-bound human polyvalent Ig. The 7 healthy individuals could thus be identified as low, intermediate or high responders depending on the concentration of immobilized human polyvalent Ig required to induce maximal NET formation.

Low responders, such as individuals 86 and 87 (see FIGS. 10A-C and 11A-C, respectively), require a concentration of 100 μg/mL of immobilized human polyvalent Ig (concentration of human polyvalent Ig in the coating solution) to exhibit maximal NET formation. In other words, for low responders, maximal NET formation is observed with a low ratio neutrophils:human polyvalent Ig.

Intermediate responders, such as individuals 88, 89 and 90 (see FIGS. 12A-C, 13A-C and 14A-C, respectively), require a concentration of 30 μg/mL of immobilized human polyvalent Ig (concentration of human polyvalent Ig in the coating solution) to exhibit maximal NET formation. In other words, for intermediate responders, maximal NET formation is observed with an intermediate ratio neutrophils:human polyvalent Ig.

High responders, such as individuals 85 and 91 (see FIGS. 15A-C and 16A-C, respectively), require a concentration of only 10 μg/mL of immobilized human polyvalent Ig (concentration of human polyvalent Ig in the coating solution) to exhibit maximal NET formation. In other words, for high responders, maximal NET formation is observed with a high ratio neutrophils:human polyvalent Ig.

Strikingly, the ratio neutrophils:human polyvalent Ig at which maximal NET formation is observed is consistent for a given individual. Indeed, the responses of each individual are similar over time, and no significant differences are observed with neutrophils isolated from three different blood samples obtained at three independent time points (time points 1, 2, and 3) as illustrated, for example, with FIGS. 10A-C, 12A-C, and 16A-C.

The results of FIGS. 10-16 thus appear to indicate that each individual is characterized by an intrinsic susceptibility to NET formation. Said susceptibility to NET formation can demonstrably be determined with an in vitro assay relying on the use of immobilized human polyvalent Ig. Of note, it may be of clinical interest to assess the susceptibility of a subject to NET formation, in particular for a subject suffering from a disease or condition to which neutrophil activation may contribute.

### Example 4: Evaluation of EET Formation by Human Eosinophils Interacting with Plate-Bound Human Polyvalent Immunoglobulins

### Materials and Methods

### Reagents

High-binding, sterile 96-well plates (Greiner Bio-One, Frickenhausen, Germany) were coated with human polyvalent Ig as described in Example 1, using a coating solution comprising human polyvalent Ig at a concentration ranging from 10 to 300 μg/mL, as indicated.

Where indicated, the SYK inhibitor BAY-61-3606, obtained from Selleckchem (reference: S7006), was added to the culture medium at a final concentration of 5 μM.

### Cells and Cell Culture

Eosinophils were isolated from the peripheral blood of healthy human donors by negative magnetic bead selection using a commercially available kit (STEMCELL Technologies—reference: 19656). Isolated eosinophils were then distributed in 96-well plates (Greiner Bio-One, Frickenhausen, Germany), in particular in 96-well plates coated with human polyvalent Ig or bovine serum albumin (negative control) as described above, at a concentration of 1.5×10⁴ cells per well, corresponding to 2×10⁵ cells/mL of culture medium. Eosinophils were then cultured in HBSS with calcium and magnesium (Gibco—reference: 24020-091) under standard conditions (i.e., humidified incubator 37° C. and 5% CO₂).

### Detection

The fluorescent, cell-impermeant, nucleic acid dye Sytox Green was added to the culture medium at a concentration of 1 μM to detect EET formation.

Time-lapse images were acquired by live cell fluorescent microscopy. Quantitative time-course measure of EET formation was performed by computer-assisted image analysis (data are expressed as GCU/well).

### Results

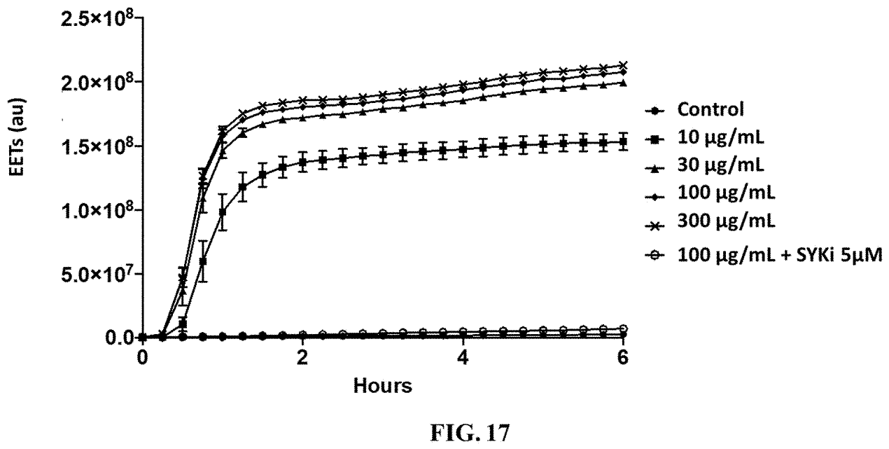

As shown on FIG. 17, EET formation is elicited by the interaction of eosinophils with immobilized polyvalent human Ig. As shown on FIG. 17, EET formation increases quickly before reaching a plateau. Of note, EET formation with a concentration of 30 μg/mL, 100 μg/mL or 300 μg/mL of human polyvalent Ig (concentration in the coating solution used to prepare the coated culture plates) reaches the same maximal level, which is higher than the maximal response obtained with a concentration of 10 μg/mL of human polyvalent Ig.

Also, as for neutrophils, EET formation by eosinophils induced by immobilized human polyvalent Ig is dependent on the SYK signaling downstream of the Fc receptors expressed at the surface of eosinophils, as demonstrated by the condition with 100 μg/mL of human polyvalent Ig and 5 μM of SYK inhibitor BAY-61-3606 in which there is no EET formation.

These results demonstrate that plate-bound human polyvalent Ig are able to induce EET formation from eosinophils, and that EET formation induced by immobilized human polyvalent Ig are dependent on the SYK signaling downstream of the Fc receptors expressed at the surface of eosinophils. Moreover, these results demonstrate that immobilized human polyvalent Ig as described herein constitute a physiological inducer of EET formation, which acts via signaling pathways that are involved in the formation of EETs in vivo.

### Example 5: Evaluation of ET Formation by Human Whole Blood Cells Interacting with Plate-Bound Human Polyvalent Immunoglobulins

### Materials and Methods

### Reagents

High-binding, sterile 96-well plates (Greiner Bio-One, Frickenhausen, Germany) were coated with human polyvalent Ig as described in Example 1, using a coating solution comprising human polyvalent Ig at a concentration ranging from 10 to 80 μg/mL, as indicated.

Where indicated, the SYK inhibitor BAY-61-3606, obtained from Selleckchem (reference: S7006), was added to the whole blood samples diluted in culture medium at a final concentration of 5 μM.

### Cells

Fresh human peripheral whole blood was collected from healthy donors and diluted ten times (1/10 dilution) in culture medium containing cell-impermeant, nucleic acid dye Sytox Green at a concentration of 1 μM to detect ET formation. Diluted whole blood was then distributed in 96-well plates (Greiner Bio-One, Frickenhausen, Germany), in particular in 96-well plates coated with human polyvalent Ig or bovine serum albumin (negative control) as described above.

### Detection

Time-lapse images were acquired by live cell fluorescent microscopy. Quantitative time-course measure of ET formation was performed by computer-assisted image analysis (data are expressed as GCU/well).

### Results

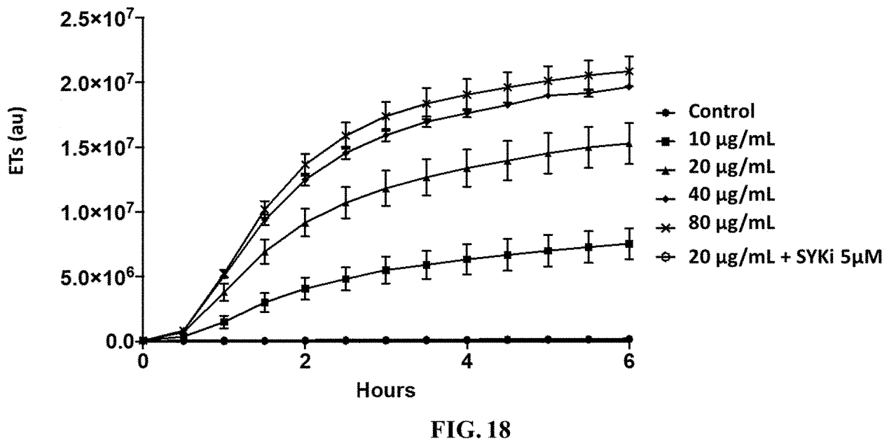

As shown on FIG. 18, ET formation is elicited by the interaction of whole blood cells with immobilized polyvalent human Ig, in a dose-dependent manner. As shown on FIG. 18, ET formation increases progressively from 10 μg/mL to 80 μg/mL of human polyvalent immunoglobulins (concentration in the coating solution used to prepare the coated culture plates).

Also, as for neutrophils and eosinophils, ET formation by whole blood immune cells induced by immobilized human polyvalent Ig is dependent on the SYK signaling downstream of the Fc receptors expressed at the surface of the cells, as demonstrated by the condition with 20 μg/mL of human polyvalent Ig and 5 μM of SYK inhibitor BAY-61-3606 in which there is no ET formation.

These results demonstrate that plate-bound human polyvalent Ig are able to induce ET formation from whole blood cells, and that ET formation induced by immobilized human polyvalent Ig are dependent on the SYK signaling downstream of the Fc receptors expressed at the surface of the cells. Moreover, these results demonstrate that immobilized human polyvalent Ig as described herein constitute a physiological inducer of ET formation, which acts via signaling pathways that are involved in the formation of ETs in vivo.

1. An in vitro method for inducing extracellular trap (ET) formation, said method comprising contacting immune cells, in in vitro culture or within a biological sample, with human polyvalent immunoglobulins, wherein said human polyvalent immunoglobulins are immobilized on a solid support.

2. The method according to claim 1, wherein said immune cells are granulocytes, macrophages and/or mast cells.

3. The method according to claim 1, wherein said immune cells are neutrophils and/or eosinophils.

4. The method according to claim 1, wherein said method does not comprise contacting the immune cells in in vitro culture or within a biological sample with phorbol-12-myristate-13-acetate (PMA).

5. The method according to claim 1, wherein the solid support is coated with human polyvalent immunoglobulins using a coating solution comprising human polyvalent immunoglobulins at a concentration of at least about 3 μg/mL.

6. The method according to claim 1, wherein the immune cells are cultured at a concentration of at least about 1×10⁵ cells/mL of culture medium or present in the biological sample at a concentration of at least about 1×10⁵ cells/mL of sample.

7. The method according to claim 1, wherein the immune cells are cultured in a culture medium comprising a cell-impermeant nucleic acid dye or wherein the biological sample is diluted in a culture medium comprising a cell-impermeant nucleic acid dye.

8. The method according to claim 7, wherein the method comprises assessing ET formation by image analysis.

9. The method according to claim 7, wherein the cell-impermeant nucleic acid dye is fluorescent and wherein the method comprises assessing ET formation by fluorescence detection.

10. An in vitro method for screening a drug for its ability to modulate extracellular trap (ET) formation, said method comprising:
a) contacting immune cells, in in vitro culture or within a biological sample, with a drug;
b) inducing ET formation by contacting the immune cells in in vitro culture or within a biological sample with human polyvalent immunoglobulins immobilized on a solid support according to the method of claim 1; and
c) assessing the effect of the drug on ET formation in the in vitro culture of immune cells or in the biological sample,
wherein the order of step a) and b) can be inverted.

11. An in vitro method for assessing extracellular trap (ET) formation in a subject, said method comprising:
a) contacting a biological sample comprising immune cells or an in vitro culture of immune cells, previously obtained from a subject, with human polyvalent immunoglobulins immobilized on a solid support, thereby inducing ET formation according to the method of claim 1; and
b) quantifying ET formation induced in the biological sample or in the in vitro culture of immune cells.

12. An in vitro method for assessing the susceptibility of a subject to extracellular trap (ET) formation, said method comprising:
a) contacting a biological sample comprising immune cells or an in vitro culture of immune cells, previously obtained from a subject, with human polyvalent immunoglobulins immobilized on a solid support, thereby inducing ET formation according to the method of claim 1; and
b) quantifying ET formation induced in the biological sample or in the in vitro culture of immune cells, thereby assessing the susceptibility of the subject to ET formation.

13. The method according to claim 12, further comprising determining the ratio of immune cells:human polyvalent immunoglobulins required to induce maximal ET formation in the biological sample comprising immune cells or in the in vitro culture of immune cells.

14. An in vitro method for predicting the response of a subject to a modulator of extracellular trap (ET) formation, said method comprising:
a) contacting a biological sample comprising immune cells or an in vitro culture of immune cells, previously obtained from a subject, with a modulator of ET formation;
b) inducing ET formation by contacting the biological sample or the in vitro culture of immune cells with human polyvalent immunoglobulins immobilized on a solid support according to the method of claim 1; and
c) assessing the effect of the modulator of ET formation on ET formation in the biological sample or in the in vitro culture of immune cells,
wherein the order of step a) and b) can be inverted.

15. The method according to claim 14, wherein the method further comprises a step of comparing the ET formation induced in the biological sample or in the in vitro culture of immune cells to a reference value, wherein the reference value is the ET formation induced in a biological sample comprising immune cells or in an in vitro culture of immune cells, previously obtained from the subject, without the modulator of ET formation.

16. The method according to claim 14, wherein the subject is suffering from a disease selected from an inflammatory disease, a thromboembolic disease, a cancer, and a fibrosis.

17. An in vitro method for monitoring the response of a subject to a therapeutic or prophylactic agent, said method comprising:
a) contacting a biological sample comprising immune cells or an in vitro culture of immune cells, previously obtained from a subject, with human polyvalent immunoglobulins immobilized on a solid support, thereby inducing ET formation according to the method of claim 1, wherein said subject was treated or is being treated with a therapeutic or prophylactic agent;
b) quantifying ET formation induced in the biological sample or in the in vitro culture of immune cells; and
c) comparing the ET formation induced in the biological sample or in the in vitro culture of immune cells to a reference value, thereby monitoring the response of the subject to the therapeutic or prophylactic agent.

18. The method according to claim 17, wherein the reference value is the ET formation induced in a biological sample comprising immune cells or in an in vitro culture of immune cells, previously obtained from the subject before treatment with the therapeutic or prophylactic agent.

19. The method according to claim 17, wherein the subject is suffering from a disease selected from an inflammatory disease, a thromboembolic disease, a cancer, and a fibrosis.
